SEQUENCE LISTING (17 sequences)

SEQ ID NO: 1 - protein, 7 aa, Artificial Sequence
  Synthetic
KTHTCPP

SEQ ID NO: 2 - protein, 233 aa, Artificial Sequence
  human TNF alpha
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 107 aa, Artificial Sequence
  VL
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 4 - protein, 119 aa, Artificial Sequence
  VH
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 5 - protein, 214 aa, Artificial Sequence
  Full length LC
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, Artificial Sequence
  Full length HC
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 1225 aa, Artificial Sequence
  NP_001005862.1 receptor tyrosine-protein kinase erbB-2 isoform b [Homo sapiens]
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 8 - protein, 6 aa, Artificial Sequence
  LC CDR1
QDVNTA

SEQ ID NO: 9 - protein, 3 aa, Artificial Sequence
  LC CDR2
SAS

SEQ ID NO: 10 - protein, 9 aa, Artificial Sequence
  LC CDR3
QQHYTTPPT

SEQ ID NO: 11 - protein, 8 aa, Artificial Sequence
  HC CDR1
GFNIKDTY

SEQ ID NO: 12 - protein, 8 aa, Artificial Sequence
  HC CDR2
IYPTNGYT

SEQ ID NO: 13 - protein, 13 aa, Artificial Sequence
  HC CDR3
SRWGGDGFYAMDY

SEQ ID NO: 14 - protein, 107 aa, Artificial Sequence [sequence omitted]

SEQ ID NO: 15 - protein, 120 aa, Artificial Sequence [sequence omitted]

SEQ ID NO: 16 - protein, 214 aa, Artificial Sequence
  Full length LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 450 aa, Artificial Sequence
  Full length HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0043501 A1
(43) Pub. Date: Feb. 8, 2024

(54) RELATIVE UNPAIRED GLYCANS IN ANTIBODY PRODUCTION METHODS

(71) Applicant: AMGEN INC., Thousand Oaks, CA (US)

(72) Inventors: Alla POLOZOVA, North Kingstown, RI (US); Chendi NIU, Rockville, MD (US); Ramsey A. SALEEM, Seattle, WA (US); Sreekanth SURAVAJJALA, Arlington, MA (US)

(73) Assignee: AMGEN INC., Thousand Oaks, CA (US)

(21) Appl. No.: 18/031,691

(22) PCT Filed: Oct. 14, 2021
(86) PCT No.: PCT/US2021/054950

§ 371 Date: Apr. 13, 2023

Related U.S. Application Data
(60) Provisional application No. 63/163,131, filed on Mar. 19, 2021.
(60) Provisional application No. 63/092,281, filed on Oct. 15, 2020.

(51) Int. Cl. C07K 16/00; C07K 1/16

(52) U.S. Cl. CPC C07K 16/00; C07K 1/16; C07K 2317/14; C07K 2317/41; C07K 2317/732

(57) ABSTRACT

Provided herein are methods of producing an antibody composition comprising determining the relative unpaired afucosylated glycan content and/or the relative unpaired high mannose glycan content of a sample of the antibody composition and selecting the antibody composition for downstream processing based on the relative unpaired afucosylated glycan content and/or relative unpaired high mannose glycan content and/or the ADCC level of the antibody composition. Related methods of determining the relative unpaired glycan content of an antibody composition and methods of modifying the ADCC level of an antibody composition are provided herein.

### CROSS REFERENCE TO RELATED APPLICATIONS

The benefit under 35 U.S.C. § 119(e) of U.S. Provisional Patent Application No. 63/092,281, filed on Oct. 15, 2020; and U.S. Provisional Patent Application No. 63/163,131, filed on Mar. 19, 2021, is hereby claimed, and the entire disclosure of each is incorporated herein by reference

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 30,284 bytes ASCII (Text) file named “A-2626-WO-PCT_Seq_Listing_ST25.bd”; created on Oct. 12, 2021.

### BACKGROUND

Glycosylation is one of the most common, yet impactful, post-translational modifications, as it plays a role in multiple cellular functions, including, for example, protein folding, quality control, molecular trafficking and sorting, and cell surface receptor interaction. Glycosylation affects the therapeutic efficacy of recombinant protein drugs, as it influences the bioactivity, pharmacokinetics, immunogenicity, solubility, and in vivo clearance of therapeutic glycoproteins. Fc glycoform profiles, in particular, are product quality attributes for recombinant antibodies, as they directly impact the clinical efficacy and pharmacokinetics of the antibodies.

Specific glycan structures associated with the conserved bi-antennary glycan in the Fc-CH2 domain can strongly influence the interaction of the Fc domain with the Fc-gamma receptors (FcγRs) that mediate antibody effector functions, e.g., antibody dependent cellular cytotoxicity (ADCC) (see Reusch D, Tejada M L. Fc glycans of therapeutic antibodies as critical quality attributes. Glycobiology 2015; 25:1325-34). For example, core fucose has been demonstrated to have a significant impact on FcγRIIIa binding affinity, leading to substantial changes in ADCC activity (see Okazaki A, et al. Fucose depletion from human IgG1 oligosaccharide enhances binding enthalpy and association rate between IgG1 and FcgammaRIIIa. Journal of molecular biology 2004; 336:1239-49; Ferrara C, et al. Unique carbohydrate-carbohydrate interactions are required for high affinity binding between FcgammaRIII and antibodies lacking core fucose. Proceedings of the National Academy of Sciences of the United States of America 2011; 108:12669-74). It has also been shown that high mannose levels also play a role in modulating ADCC activity, though to a much more modest and less predictable extent than core fucose (Thomann M, et al. Fc-galactosylation modulates antibody-dependent cellular cytotoxicity of therapeutic antibodies. Molecular immunology 2016; 73:69-75). Because core fucose has been reported to sterically hinder the Fc domain from interacting with the FcγR, much research has focused on glycan groups which lack core fucose, including afucosylated glycans and high mannose glycans.

The existing knowledge of Fc-FcγRIIIa interactions focuses on FcγRIIIa binding to a single Fc chain. However, as protein therapeutic molecules, including IgG and fusion proteins with IgG Fc region, typically contain two glycosylated Fc chains, the effect of glycan pairing on FcγRIIIa binding interactions and resulting ADCC activity are unknown. Different factors influence the glycan structure and thus the ultimate glycosylated form (glycoform) of the protein (glycoprotein). For example, the cell line expressing the antibody, the cell culture medium, the feed medium composition, and the timing of the feeds during cell culture can impact the production of glycoforms of the protein. While research groups have suggested many ways to influence the levels of particular glycoforms of an antibody, there still is a need in the biopharmaceutical industry for simple, efficient and reliable methods of predicting the effector function of a particular antibody composition based on the given glycoform profile for that antibody composition.

### SUMMARY

Presented herein are data supporting that the unpaired glycan content of an antibody composition is related to the ADCC activity level for the antibody composition. The data also support that the ADCC activity level of the antibody composition may be modified by changing the unpaired glycan content of the antibody composition. Without being bound to a particular theory, the unpaired afucosylated glycan content and/or the unpaired high mannose glycan content of an antibody composition is related to the ADCC activity level of the antibody composition, and, changing the unpaired afucosylated glycan content and/or the unpaired high mannose glycan content of the antibody composition leads to modification of the ADCC activity level of the antibody composition.

Accordingly, the present disclosure provides methods of modifying the ADCC activity level of an antibody composition. In exemplary embodiments, the method comprises modifying the relative unpaired afucosylated glycan content of an antibody composition and/or the relative unpaired high mannose glycan content of an antibody composition. In exemplary aspects, the method comprises increasing the relative unpaired afucosylated glycan content to increase the level of ADCC activity. In exemplary instances, the method comprises increasing the relative unpaired high mannose glycan content to increase the level of ADCC activity. In various aspects, the method comprises decreasing the relative unpaired afucosylated glycan content to decrease the level of ADCC activity. In various instances, the method comprises decreasing the relative unpaired high mannose glycan content to decrease the level of ADCC activity. In exemplary aspects, the level of ADCC activity is modified by about 10% to about 15%, when the relative unpaired afucosylated glycan content or the relative unpaired high mannose glycan content is modified by about 1%. For every 1% increase in relative unpaired afucosylated glycan content or relative unpaired high mannose glycan, in some aspects, the level of ADCC activity is increased by about 10% to about 15%. For every 1% decrease in relative unpaired afucosylated glycan content or relative unpaired high mannose glycan, in some aspects, the level of ADCC activity is decreased by about 10% to about 15%. In some aspects, the level of ADCC activity is modified by the sum of about 10% to about 15% times the relative unpaired afucosylated glycan content plus about 10% to about 15% times the relative unpaired high mannose glycan content. Optionally, the level of ADCC activity is modified by the sum of about 12% to about 13% times the relative unpaired afucosylated glycan content plus about 12.5% to about 13.5% times the relative unpaired high mannose glycan content. In various aspects, the antibody of the antibody composition is an anti-TNF antibody, optionally, infliximab, adalimumab, golimumab, or certolizumab pegol. In alternative aspects, the level of ADCC activity is modified by about 10% to about 15%, when the relative unpaired afucosylated glycan content is modified by about 1% and the antibody of the antibody composition is an anti-HER2 antibody, optionally, trastuzumab or pertuzumab. For every 1% increase in relative unpaired afucosylated glycan content, in some aspects, the level of ADCC activity is increased by about 10% to about 15%. For every 1% decrease in relative unpaired afucosylated glycan content, in some aspects, the level of ADCC activity is decreased by about 10% to about 15%. In various instances, the level of ADCC is not modified upon a change in relative unpaired high mannose glycan content.

The present disclosure also provides methods of producing an antibody composition. In exemplary embodiments, the method comprises (i) determining the relative unpaired afucosylated glycan content and/or the relative unpaired high mannose glycan content of a sample of the antibody composition; and (ii) selecting the antibody composition for downstream processing based on the relative unpaired afucosylated glycan content and/or relative unpaired high mannose glycan content determined in (i). In various aspects, the sample is taken from a cell culture comprising cells, e.g., glycosylation-competent cells, expressing an antibody of the antibody composition. Optionally, the method further comprises modifying one or more conditions of the cell culture to modify the relative unpaired afucosylated glycan content and/or the relative unpaired high mannose glycan content of the antibody composition and determining the relative unpaired afucosylated glycan content and/or the relative unpaired high mannose glycan content of a sample of the modified cell culture. In exemplary aspects, the method comprises repeating the modifying until the relative unpaired afucosylated glycan content and/or relative unpaired high mannose glycan content is within a target range. In exemplary aspects, the target range is based on a target range of ADCC activity levels for a reference antibody and a model which correlates ADCC activity level of the antibody composition to afucosylated glycan content and/or high mannose glycan content of the antibody composition, optionally, a model which correlates ADCC activity level of the antibody composition to relative unpaired afucosylated glycan content and/or relative unpaired high mannose glycan content of the antibody composition. In exemplary instances, the reference antibody is infliximab. In exemplary aspects, the reference antibody is trastuzumab. Optionally, the target range of the relative unpaired afucosylated glycan content is about 80% to about 90% and/or the target range of the relative unpaired high mannose glycan is about 75% to about 85%, when the antibody is an anti-TNF antibody, such as, infliximab. Optionally, the target range of the relative unpaired afucosylated glycan content is about 95% to about 99% and/or the target range of the relative unpaired high mannose glycan is below about 25% to about 35%, when the antibody is an anti-HER2 antibody, such as, trastuzumab. In various aspects, the relative unpaired afucosylated glycan content and/or the relative unpaired high mannose glycan content is/are determined in real time with respect to production of the antibody composition. In various instances, the method comprises selecting the antibody composition for downstream processing when the relative unpaired afucosylated glycan content and/or relative unpaired high mannose glycan content is/are in a target range. In various aspects, the relative unpaired afucosylated glycan content and/or relative unpaired high mannose glycan content correlate with the ADCC activity level of the antibody composition. Optionally, the method further comprises determining the ADCC activity level of the antibody composition based on the relative unpaired afucosylated glycan content and/or relative unpaired high mannose glycan content determined in (i). Optionally, the method comprises selecting the antibody composition for downstream processing when the ADCC activity level is in a target range. In some embodiments, selecting the antibody composition for downstream processing comprises selecting a clone that produces antibody composition with a specified level of relative unpaired afucosylated glycan content and/or relative unpaired high mannose glycan content.

In exemplary embodiments, the method of producing an antibody composition comprises (i) determining the relative unpaired afucosylated glycan content of an antibody composition and/or the relative unpaired high mannose glycan content of an antibody composition; (ii) determining the ADCC level of the antibody composition based on the relative unpaired afucosylated glycan content and/or the relative unpaired high mannose glycan content determined in (i); and (iii) selecting the antibody composition for downstream processing when the ADCC level of the antibody composition determined in (ii) is within a target ADCC range.

In exemplary embodiments, the method of producing an antibody composition comprises (i) determining the relative unpaired afucosylated glycan content and/or the relative unpaired high mannose glycan content of a sample of the antibody composition taken from a cell culture comprising glycosylation-competent cells expressing an antibody of the antibody composition; (ii) optionally, modifying the cell culture to modulate the relative unpaired afucosylated glycan content and/or the relative unpaired high mannose glycan content and determining the relative unpaired afucosylated glycan content and/or the relative unpaired high mannose glycan content of a sample of the antibody composition taken from the modified cell culture; and (iii) selecting the antibody composition for downstream processing based on the relative unpaired afucosylated glycan content and/or relative unpaired high mannose glycan content.

In various aspects of the presently disclosed methods of producing an antibody composition, the method comprises modifying the ADCC level of an antibody composition according to those methods of the present disclosure. In various instances, the method comprises determining the relative unpaired glycan content of an antibody composition by treating the antibody composition with an enzyme which cleaves an antibody heavy chain at a site N-terminal to the hinge region disulfide linkages to form antibody fragments, (ii) separating the antibody fragments by a chromatography, and (iii) quantifying each antibody fragment. Optionally, the site is between Thr and His or between Lys and Thr of the sequence KTHTCPP (SEQ ID NO: 1) of an IgG1 antibody heavy chain. In various aspects, the antibody fragments comprise Fab fragments and glycosylated Fc fragments. In various instances, the antibody fragments are separated through hydrophilic interaction liquid chromatography (HILIC). In some aspects, the quantifying of each antibody fragment comprises mass spectrometry. Optionally, the glycosylated Fc fragments comprise Fc fragments attached to one of a variety of glycan moieties, and the method comprises separating and quantifying glycosylated Fc fragments according to the attached glycan moiety. In exemplary aspects, the method comprises selecting the antibody composition for downstream processing comprises selecting a clone that produces the antibody composition having a selected relative unpaired afucosylated glycan content and/or relative unpaired high mannose glycan content.

The present disclosure additionally provides methods of determining the relative unpaired glycan content of an antibody composition, comprising (i) treating the antibody composition with an enzyme which cleaves an antibody heavy chain at a site N-terminal to the hinge region disulfide linkages to form antibody fragments, (ii) separating the antibody fragments by a chromatography, and (iii) quantifying each antibody fragment. In various aspects, the enzyme is a cysteine protease. In various instances, the enzyme is a member of the IgdE protease family, optionally, an IgdE expressed by a Streptococcus. In various instances, the enzyme is structurally identical or highly similar to an IgdE protease expressed by Streptococcus agalactiae. In various aspects, the enzyme is structurally identical or highly similar to an enzyme expressed by a Porphyromonas anaerobe. In various instances, the enzyme is structurally identical or highly similar to an enzyme expressed by a Porphyromonas gingivalis. In exemplary aspects, the enzyme is a gingipain K (which may also be referred to “Kgp”). In various instances, the site is between Thr and His or between Lys and Thr of the sequence KTHTCPP (SEQ ID NO: 1) of an IgG1 antibody heavy chain. In various aspects, the antibody fragments comprise Fab fragments and glycosylated Fc fragments. In exemplary aspects, the separating of (ii) comprises hydrophilic interaction liquid chromatography (HILIC). In exemplary aspects, the quantifying of (iii) comprises mass spectrometry. In various instances, the glycosylated Fc fragments comprise Fc fragments attached to one of a variety of glycan moieties, and the method comprises separating and quantifying glycosylated Fc fragments according to the attached glycan moiety. In various aspects, the relative unpaired afucosylated glycans and/or relative unpaired high mannose glycans of the antibody composition is/are quantified.

### BRIEF DESCRIPTION OF THE DRAWINGS

FIG. 1A is an illustration of exemplary glycan structures. FIG. 1B is and illustration of exemplary glycan groups.

FIG. 2A is a representative glycan map chromatogram (full scale view; y-axis max˜440.00 EU)). FIG. 2B is a representative glycan map chromatogram (expanded scale view; y-axis max˜44.00 EU).

FIG. 3 is a diagram of the salvage pathway and the de novo pathway of fucose metabolism. In the salvage pathway, free L-fucose is converted to GDP-fucose, while in the de novo pathway, GDP-fucose is synthesized via three reactions catalyzed by GMD and FX. GDP-fucose is then transported from the cytosol to the Golgi lumen by GDP-Fuc Transferase and transferred to acceptor oligosaccharides and proteins. The other reaction product, GDP, is converted by a luminal nucleotide diphosphatase to guanosine 5-monophosphate (GMP) and inorganic phosphate (Pi). The former is exported to the cytosol (via an antiport system that is coupled with the transport of GDP-fucose), whereas the latter is postulated to leave the Golgi lumen via the Golgi anion channel, GOLAC. See, e.g., Nordeen et al. 2000; Hirschberg et al. 2001.

FIGS. 4A-4D are drawings of exemplary antibodies with unpaired or paired glycans. FIG. 4A is a drawing of unpaired afucosylated glycan A2G1. FIG. 4B is a drawing of paired A2G1 glycans. FIG. 4C is a drawing of unpaired high mannose 5 (HM5) glycans. FIG. 4D is a drawing of paired HM5 glycans.

FIG. 5 is an illustration of an antibody before and after treatment with FabALACTICA overnight at 37° C. Chromatographic separation with MS detection was carried out on the FabALACTICA-digested material to separate the Fab fragments and glycosylated Fc fragments.

FIG. 6A is a pairing glycan map chromatogram of Antibody A and FIG. 6B is a pairing glycan map chromatogram of Antibody B.

FIG. 7 is a graph of the relative abundance of unpaired afucosylated glycans (%) and relative abundance of unpaired high mannose glycans (%) for Antibody A and Antibody B. The relative abundance of unpaired afucosylated glycans (%) was calculated by dividing the percentage of unpaired afucosylated glycans by the sum of the percentage of unpaired afucosylated glycans and the percentage of paired afucosylated glycans and multiplying by 100%. The relative abundance of unpaired high mannose glycans was calculated by dividing the percentage of unpaired high mannose glycans by the sum of the percentage of unpaired high mannose glycans and the percentage of paired high mannose glycans and multiplying by 100%.

FIG. 8A is a schematic of a cell-based ADCC assay. FIG. 8B is a representative dose-response curve for the NK92 ADCC Assay for Antibody A. Each dose point is a mean±standard deviation of 3 replicates. Assay signal=fluorescence

FIG. 9A is leverage plot of relative ADCC activity level (%) of Antibody A as measured by the cell-based ADCC assay plotted as a function of measured afucosylated glycan content (%) of Antibody A. The best fit line is the solid diagonal line in the middle of the shaded area. p<0.0001.

FIG. 9B is leverage plot of relative ADCC activity level (%) of Antibody A as measured by the cell-based ADCC assay plotted as a function of measured high mannose glycan content (%) of Antibody A. The best fit line is the solid diagonal line in the middle of the shaded area. p=0.0009.

FIG. 9C is graph of the Actual ADCC activity level (%) of Antibody A as measured by the cell-based ADCC assay plotted against the Predicted ADCC activity level (%) of Antibody A as calculated using Equation 1.

FIG. 10A is leverage plot of relative ADCC activity level (%) of Antibody B as measured by the cell-based ADCC assay plotted as a function of measured afucosylated glycan content (%) of Antibody B. The best fit line is the solid diagonal line in the middle of the shaded area. p<0.0001.

FIG. 10B is leverage plot of relative ADCC activity level (%) of Antibody B as measured by the cell-based ADCC assay plotted as a function of measured high mannose glycan content (%) of Antibody B. The best fit line is the solid diagonal line in the middle of the shaded area. p=0.3263.

FIG. 10C is graph of the Actual ADCC activity level (%) of Antibody B as measured by the cell-based ADCC assay plotted against the Predicted ADCC activity level (%) of Antibody B as calculated using Equation 2.

FIG. 11 is a graph of the measured ADCC activity level (%) plotted as a function of measured FcγRIIIa binding for Antibody B.

FIG. 12 is a deconvoluted mass spectra from the intact mass analysis of Antibody B, wherein the peaks for the indicated glycan pairs are shown.

FIG. 13 is an illustration of an antibody before and after treatment with GingisKHAN for 60 min at 37 degrees C. Chromatographic separation with MS detection was carried out on the GingisKHAN-digested material to separate the Fab fragments and glycosylated Fc fragments

FIG. 14A is a graph showing exemplary results from the separation and detection described in FIG. 13. FIG. 14B is an expanded view of the peaks of the Fc fragments with labeled glycan pairs.

FIG. 15 is an example of extracted ion chromatograms showing elution of individual Fc glycan pairs species.

FIG. 16A is leverage plot of measured FcγRIIIa binding (%) of Antibody C plotted as a function of measured total abundance of afucosylated glycan content (%) of Antibody C. The best fit line is the solid diagonal line in the middle of the shaded area. p=0.1838. FIG. 16B is leverage plot of measured FcγRIIIa binding (%) of Antibody C plotted as a function of measured total abundance of high mannose glycan content (%) of Antibody C. The best fit line is the solid diagonal line in the middle of the shaded area. p=0.1086. FIG. 16C is graph of the Actual (measured) FcγRIIIa binding (%) of Antibody C plotted against the Predicted FcγRIIIa binding (%) of Antibody C as calculated based on total abundance of afucosylated glycan content and total abundance of high mannose glycan content using Equation 3. RMSE=16.797; r 2=0.45; p=0.0679.

FIG. 17A is leverage plot of measured FcγRIIIa binding (%) of Antibody C plotted as a function of relative abundance of unpaired afucosylated glycans (%) of Antibody C. The best fit line is the solid diagonal line in the middle of the shaded area. p=0.0022. FIG. 17B is leverage plot of measured FcγRIIIa binding (%) of Antibody C plotted as a function of relative abundance of unpaired high mannose glycans (%) of Antibody C. The best fit line is the solid diagonal line in the middle of the shaded area. p=0.0099. FIG. 17C is graph of the Actual (measured) FcγRIIIa binding (%) of Antibody C plotted against the Predicted FcγRIIIa binding (%) of Antibody C as calculated based on unpaired afucosylated and unpaired high mannose glycans using Equation 4. RMSE=13.071; r²=0.67; p=0.0071.

### DETAILED DESCRIPTION

Described herein are methods of determining the relative unpaired glycan content of proteins such as antibodies, and methods of modifying the ADCC level of an antibody composition. Data described herein suggest that the relative unpaired glycan content of an antibody composition is correlative with the ADCC activity level for the antibody composition and that the ADCC activity level of the antibody composition may be modified by modifying the relative unpaired glycan content of the antibody composition. It is further contemplated that a higher relative unpaired afucosylated glycan content and/or relative unpaired high mannose content has greater leverage on ADCC activity levels than relative paired glycan content, so that a percent change in relative unpaired glycan content will have a greater effect on ADCC activity levels than the same percent change in relative paired glycan content. Accordingly, provided herein are methods of modifying the ADCC level of an antibody composition. The methods can comprise producing an antibody composition having a selected relative unpaired afucosylated glycan content and/or selected relative unpaired high mannose glycan content in order to achieve a selected level of ADCC. For example, in accordance with methods described herein, a clone producing an antibody composition having a selected relative unpaired afucosylated glycan content and/or selected relative unpaired high mannose glycan content, can be chosen or selected. An antibody composition can be produced from the clone.

Glycosylation, Glycans, and Methods of Glycan Measurement

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or microheterogeneity of protein glycoforms exists due to the large range of glycan structures associated with each site (0 or N).

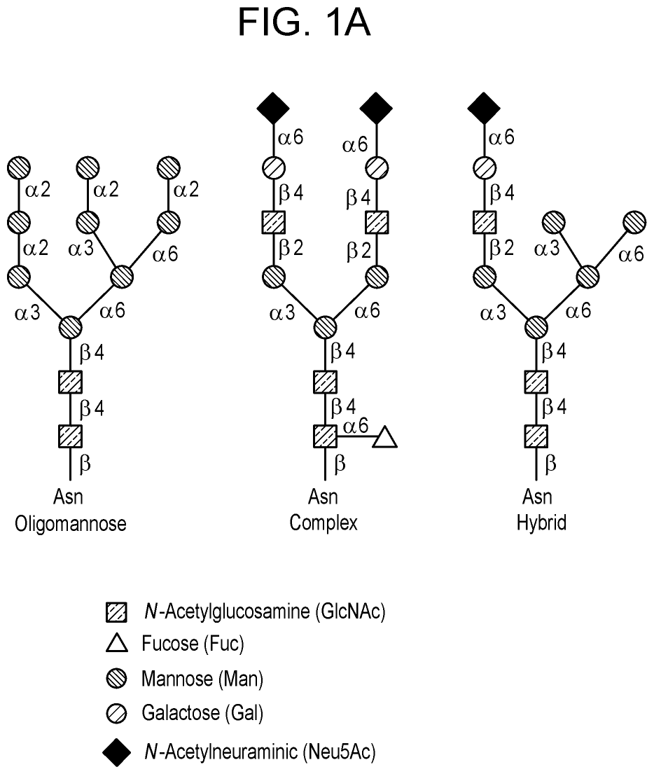

All N-glycans have a common core sugar sequence: Manα1-6(Manα1-3)Manβ1-4G1cNAcβ1-4G1cNAcβ1-Asn-X-Ser/Thr (Man₃G1cNAc 2 Asn) and are categorized into one of three types: (A) a high mannose (HM) or oligomannose (OM) type, which consists of two N-acetylglucosamine (GalNAc) moieties and at least 5 (e.g., 5, 6, 7, 8 or 9) mannose (Man) residues, (B) a complex type, which comprises more than two GlcNAc moieties and any number of other sugar types, or (C) a hybrid type, which comprises a Man residue on one side of the branch and GlcNAc at the base of a complex branch. FIG. 1A (adapted from Stanley et al., Chapter 8: N-Glycans, Essentials of Glycobiology, 2^{nd} ed., Cold Spring Harbor Laboratory Press; 2009) shows the three types of N-glycans.

N-linked glycans found in IgG molecules typically comprise one or more monosaccharides of galactose (Gal), N-glucose (GLc), N-acetylglucoasamine (ClcNAc), glucoasamine (GlcN), mannose (Man), fucose (Fuc), Exemplary glycans, their identity and group classifications are shown in FIG. 1B.

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

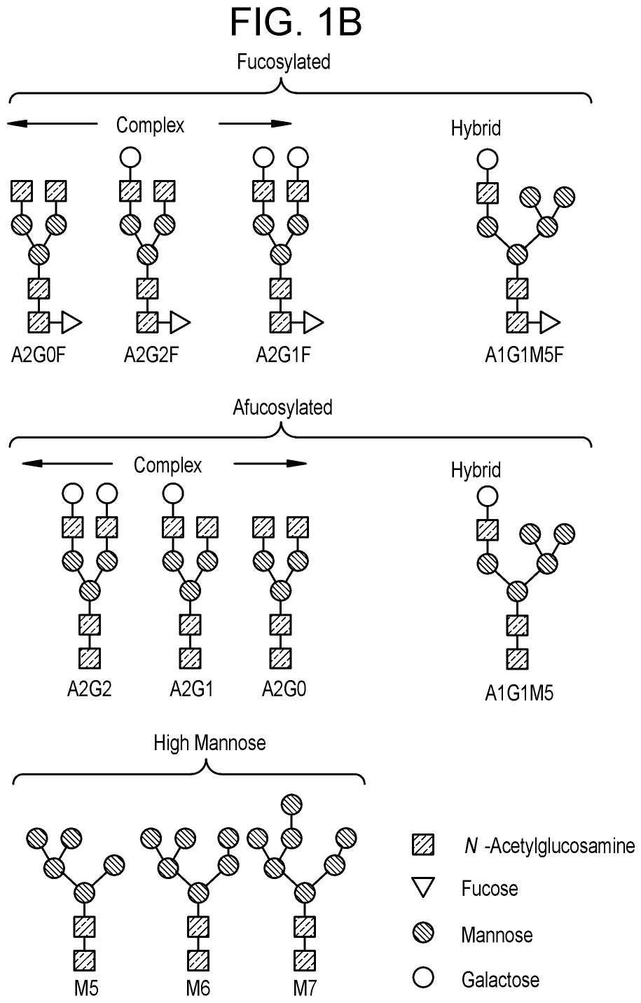

Various methods may be used for assessing glycans present in a glycoprotein-containing composition or for determining, detecting or measuring a glycoform profile (e.g., a glycoprofile) of a particular sample comprising glycoproteins. Suitable methods include, but are not limited to, positive ion MALDI-TOF analysis, negative ion MALDI-TOF analysis, weak anion exchange (WAX) chromatography, normal phase chromatography (NP-HPLC), exoglycosidase digestion, Bio-Gel P-4 chromatography, anion-exchange chromatography and one-dimensional n.m.r. spectroscopy, and combinations thereof. See, e.g., Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481 and Geoffrey, R. G. et. al. Analytical Biochemistry 1996, Vol. 240, pages 210-226. Also, Example 1 set forth herein describes a suitable method for assessing (e.g., determining, identifying, quantifying) glycans present in a glycoprotein-containing composition, e.g., an antibody composition. This method may not be used to detect whether glycans are paired or unpaired. The method of Example 1 describes an assay in which glycans attached to glycosylated proteins of a composition, e.g., antibodies of an antibody composition, are enzymatically cleaved from the protein (e.g., antibody). The glycans are subsequently separated by Hydrophilic Interaction Liquid Chromatography (HILIC) and a chromatogram with several peaks is produced. Each peak of the chromatogram represents a distribution (amount or abundance) of a different glycan. Two views of a representative HILIC chromatogram comprising peaks for different glycans are provided in FIGS. 2A and 2B. For these purposes, % Peak Area=Peak Area/Total Peak Area×100%. Accordingly, the level of a particular glycan (or groups of glycans) is reported as a %. For example, if an antibody composition is characterized as having a Man6 level of 30%, it is meant that 30% of all glycans cleaved from the antibodies of the composition are Man6. As described in more detail herein, it is noted that conventional methods that remove glycans from proteins and then analyze the composition of the glycans may identify a distribution of glycan content for a protein. However, such methods do not provide information relating to paired glycans and/or unpaired glycans.

The present disclosure relates to high mannose glycans and afucosylated glycans of an antibody composition (see FIG. 1B for examples). As used herein, the term “high mannose glycans” or “HM glycans” encompasses glycans comprising 5, 6, 7, 8, or 9 mannose residues, abbreviated as Man5 or M5, Man6 or M6, Man7 or M7, Man8 or M8, and Man9 or M9, respectively. A level of HM glycans, in various aspects, is obtained by summing the % Man5, the % Man6, the % Man7, the % Man8, and the % Man9. As used herein, the term “afucosylated glycan” or “AF glycan” refers to glycans which lack a core fucose, e.g., an α1,6-linked fucose on the GlcNAc residue involved in the amide bond with the Asn of the N-glycosylation site. Afucosylated glycans include, but are not limited to, A1G0, A2G0, A2G1 (a and b), A2G2, and A1G1M5. It is noted that high mannose glycans also lack core fucose (and thus represent a subset of afucosylated glycans), but high mannose glycans have certain characteristics and may be referred to as a separate glycan group. Accordingly, unless explicitly stated otherwise, high mannose is understood to represent a separate characteristic and may be classified separately from, or as an additional characteristic of afucosylated glycans. See, e.g., Reusch and Tejada, Glycobiology 25(12): 1325-1334 (2015). A level of afucosylated glycans, in various aspects, is obtained by summing the % A1G0, the % A2G0, the % A2G1a, the % A2G1b, the % A2G2, the % A1G1M5, the % A1G1a.

In exemplary aspects, the level (e.g., amount, abundance) of glycans (e.g., % HM glycans, % AF glycans) is determined (e.g., measured) by any of the various methods known in the art for assessing glycans present in a glycoprotein-containing composition or for determining, detecting or measuring a glycoform profile (e.g., a glycoprofile) of a particular sample comprising glycoproteins. In exemplary instances, the level (e.g., amount, abundance) of glycans (e.g., % HM glycans, % AF glycans) of an antibody composition is determined by measuring the level (e.g., amount, abundance) of such glycans in a sample of the antibody composition though a chromatography-based method, e.g., HILIC, and the level (e.g., amount, abundance) of glycans is expressed as a %, as described herein. See, e.g., Example 1. In exemplary instances, the level of glycans of an antibody composition is expressed as a % of all glycans cleaved from the antibodies of the composition. In various aspects, the level (e.g., amount, abundance) of glycans (e.g., % HM glycans, % AF glycans) is determined (e.g., measured) by measuring the level of such glycans in a sample of the antibody composition. In exemplary instances, samples of an antibody composition are taken and the level (e.g., amount, abundance) of glycans (e.g., % HM glycans, % AF glycans) for each sample is determined (e.g., measured). In various aspects, the % HM glycans and/or % AF glycans is determined.

Glycan Pairing and Methods of Measuring Unpaired Glycans of an Antibody Composition

In various instances, the glycoprotein comprises two polypeptide chains, and, in various aspects, each polypeptide chain is glycosylated. For example, the glycoprotein may comprise two Fc chains of an antibody and each Fc chain is covalently bound to a glycan. For example, the glycoprotein may be an antibody comprising two heavy chains, each of which comprises a glycosylated Fc region. In various aspects, the glycan attached to one Fc chain is in a different category from the glycan attached to the other Fc chain. In alternative instances, the glycan attached to one Fc chain is the same, or in the same glycan category, as the glycan attached to the other Fc chain. When the glycan attached to one Fc chain is the same or in the same glycan category, as the glycan attached to the other Fc chain, the glycans are considered as “paired”. In various instances, “paired” glycans refers to the glycans on each Fc chain being (a) identical glycans (e.g., structurally identical glycans attached to each Fc chain) or (b) non-identical glycans which fall within the same glycan category (e.g., structurally non-identical glycans attached to each Fc chain in which the glycans fall into the same glycan category, for example two afucosylated glycans, or as another example, two fucosylated glycans). When the glycans attached to the Fc chains fall into different glycan categories, the glycans are considered as “unpaired” (e.g. an afucosylated glycan and a fuscosylated glycan). In various aspects, “unpaired” means that the glycans attached to the Fc chains are not paired.

The “glycan category”, as used herein, is determined by the presence or absence of a core fucose, which may be referred to as “fucosylated” and “afucosylated,” respectively. When the glycan attached to one Fc chain is afucosylated (i.e., lacks a core fucose) and the glycan attached to the other Fc chain comprises a core fucose, the glycans are considered as “unpaired afucosylated glycans”. For unpaired afucosylated glycoproteins, the glycan groups have different fucosylation statuses among the Fc chains, of which only one Fc chain is covalently bound to a glycan that lacks a core fucose (e.g. an afucosylated glycan on the first Fc chain and a fuscosylated glycan on the second Fc chain). An “unpaired afucosylated” status may be assigned to those pairs wherein only one Fc chain has a core fucose (F), and a “paired afucosylated” status may be assigned to those pairs wherein neither Fc chain has a core fucose. Unpaired afucosylated glycans include, for instance, A1G0, A2G0, A2G1a, A2G1b, or A2G2 on one Fc chain and a glycan comprising a core fucose on the other Fc chain. “Paired afucosylated glycans” refers to having (a) identical afucosylated glycans on each Fc chain (e.g., afucosylated glycans of identical structure) or (b) non-identical afucosylated glycans (e.g., afucosylated glycans having different structures) on each Fc chain. Paired afucosylated glycans include, for instance, any of A1G0, A2G0, A2G1a, A2G1b, or A2G2 on each Fc chain. Paired afucosylated glycans include, for example, (i) A1G0 on one Fc chain and A2G0, A2G1a, A2G1b, or A2G2 on the other Fc chain or (ii) A2G0 on one chain and A2G1a, A2G1b, or A2G2 on the other Fc chain or (iii) A2G1a on one Fc chain and A2G1b or A2G2 on the other Fc chain or (iv) A2G1b on one Fc chain and A2G2 on the other chain. Paired afucosylated glycans also include, for example, identical non-high mannose glycans on each Fc chain (e.g., Man3 on each Fc chain) that each lack a core fucose, or a non-high mannose glycan that lacks a core mannose on one Fc chain and another afucosylated glycan which lacks non-high mannose (e.g., A1G0, A2G0, A2G1a, A2G1b, or A2G2) on the other Fc chain. A “paired fucosylated” status may be assigned to those pairs wherein each Fc chain has a core fucose.

In exemplary instances, the glycans (e.g., paired afucosylated glycans, unpaired afucosylated glycans) are further characterized by the presence or absence of a high mannose. For example, when one or both glycans of “paired afucosylated glycans” comprise a high mannose, the glycans are characterized as “paired high mannose glycans”. Also, for example, when one or both glycans of the “unpaired afucosylated glycans” comprises a high mannose, the glycans are characterized as “unpaired high mannose glycans”. Exemplary paired high mannose glycans include glycans having (a) identical high mannose glycans on each Fc chain (e.g., high mannose of identical structure), such as Man5, Man6, Man7, Man8 or Man9 on each chain, or (b) non-identical high mannose glycans (e.g., high mannose glycans having different structures) but each high mannose glycan comprises Man5, Man6, Man7, Man8 or Man9 (e.g., Man5 on one Fc chain and Man6, Man7, Man8, or Man9 on the other Fc chain, or Man6 on one Fc chain and Man7, Man8, or Man9 on the other Fc chain, or Man7 on one chain and Man8 or Man9 on the other Fc chain, or Man8 on one Fc chain and Man9 on the other Fc chain). Exemplary unpaired high mannose glycans include glycans having Man5, Man6, Man7, Man8 or Man9 on one chain and a fucosylated glycan on the other chain.

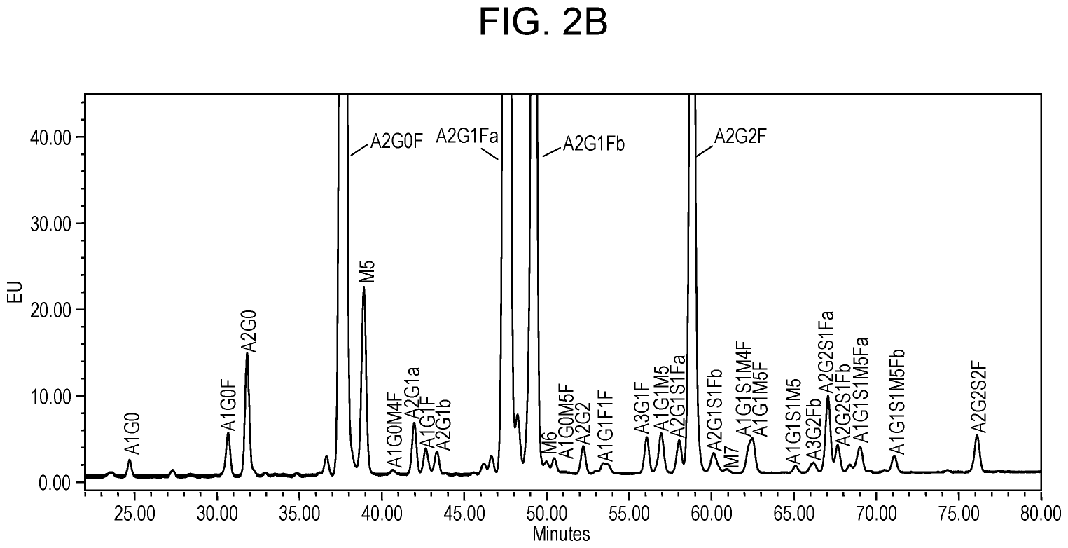

Examples of “paired” and “unpaired” glycans are provided in Tables 3 and 4. Further examples of “paired” and “unpaired” glycans are shown in FIGS. 4A-4D, wherein FIG. 4A illustrates an antibody with unpaired afucosylated glycans, FIG. 4C illustrates an antibody with unpaired high mannose glycans (because the glycans are unpaired, and one of the glycans is high mannose), FIG. 4B illustrates an antibody with paired afucosylated glycan and FIG. 4D illustrates an antibody with paired high mannose glycans.

A composition comprising the glycoprotein, e.g., an antibody composition, may be characterized in terms of its paired glycan content and its unpaired glycan content. For example, an antibody composition may be characterized in terms of its paired afucosylated glycan content and unpaired unfucosylated glycan content and/or its paired high mannose content and unpaired high mannose content.

The abundance of glycans as described herein may be referred to as relative or absolute abundance. In exemplary instances, the absolute content of glycans may be expressed in units measuring levels of the glycans themselves, for example in terms of mass, moles, mass or molar units per volume unit, arbitrary units, or area under curve (e.g., as may be determined from a chromatograph). In exemplary instances, a glycoprotein, or a composition comprising the same, is characterized in terms of its relative abundance of unpaired glycans, meaning that the amount of unpaired glycans is expressed as an amount relative to the sum of paired glycans and unpaired glycans of the glycoprotein, or composition thereof. In exemplary aspects, the glycoprotein, or a composition comprising the same, is characterized in terms of its relative abundance of unpaired afucosylated glycans. In exemplary aspects, the glycoprotein, or a composition comprising the same, is characterized in terms of its relative abundance of unpaired high mannose glycans. The term “relative abundance of unpaired afucosylated glycans” which is synonymous with “relative unpaired afucosylated glycan content” and “relative % unpaired afucosylated glycans” is calculated as dividing the percentage of unpaired afucosylated glycans by the sum of the percentage of unpaired afucosylated glycans and the percentage of paired afucosylated glycans) and multiplying by 100%. The term “relative abundance of unpaired high mannose glycans” which is synonymous with “relative unpaired high mannose glycan content” and “relative % unpaired high mannose glycans” is calculated as the percentage of unpaired high mannose glycans divided by the sum of the percentage of unpaired high mannose glycans and the percentage of paired high mannose glycans) multiplied by 100%.

The term “relative abundance of paired afucosylated glycans” is synonymous with “relative paired afucosylated glycan content” and “relative % paired afucosylated glycans” is calculated as dividing the percentage of paired afucosylated glycans by the sum of the percentage of unpaired afucosylated glycans and the percentage of paired afucosylated glycans) and multiplying by 100%. The term “relative abundance of paired high mannose glycans” is synonymous with “relative paired high mannose glycan content” and “relative % paired high mannose glycans” is calculated as dividing the percentage of paired afucosylated glycans by the sum of the percentage of unpaired afucosylated glycans and the percentage of paired afucosylated glycans) and multiplying by 100%. In various aspects of the present disclosure, the sum of the relative % unpaired glycans of a glycan category and the relative % paired glycans of the glycan category equals 100%. In various aspects of the present disclosure, the sum of the relative % unpaired afucosylated glycans and the relative % paired afucosylated glycans equals 100%. Accordingly, in various aspects, if the relative % paired afucosylated glycans is known, the relative % unpaired afucosylated glycans may be determined (e.g., calculated) by subtracting the relative % paired afucosylated glycans from 100%. Also, in various instances, if the relative % unpaired afucosylated glycans is known, the relative % paired afucosylated glycans may be determined (e.g., calculated) by subtracting the relative % unpaired afucosylated glycans from 100%. In various aspects of the present disclosure, the sum of the relative % unpaired high mannose glycans and the relative % paired high mannose glycans equals 100%. Accordingly, in various aspects, if the relative % paired high mannose glycans is known, the relative % unpaired high mannose glycans may be determined (e.g., calculated) by subtracting the relative % paired high mannose glycans from 100%. Also, in various instances, if the relative % unpaired high mannose glycans is known, the relative % paired high mannose glycans may be determined (e.g., calculated) by subtracting the relative % unpaired high mannose glycans from 100%.

The present disclosure provides methods of determining the relative unpaired glycan content of an antibody composition. In exemplary embodiments, the method comprises (i) treating the antibody composition with an enzyme which cleaves an antibody heavy chain at a site N-terminal to the hinge region disulfide linkages to form antibody fragments, (ii) separating the antibody fragments by a chromatography, and (iii) quantifying each antibody fragment. In various aspects, the enzyme is a cysteine protease. In various instances, the enzyme is a member of the IgdE protease family, optionally, an IgdE expressed by a Streptococcus. In various instances, the enzyme is structurally identical or highly similar to an IgdE protease expressed by Streptococcus agalactiae. In various aspects, the enzyme is structurally identical or highly similar to an enzyme expressed by a Porphyromonas anaerobe. In various instances, the enzyme is structurally identical or highly similar to an enzyme expressed by a Porphyromonas gingivalis. In various instances, the site is between Thr and His or between Lys and Thr of the sequence KTHTCPP (SEQ ID NO: 1) of an IgG1 antibody heavy chain. In various aspects, the antibody fragments comprise Fab fragments and glycosylated Fc fragments. In exemplary aspects, the method comprises separating the antibody fragments by hydrophilic interaction liquid chromatography (HILIC). In exemplary aspects, the method comprises quantifying each antibody fragment by mass spectrometry. In various instances, the glycosylated Fc fragments comprise Fc fragments attached to one of a variety of glycan moieties, and the method comprises separating and quantifying glycosylated Fc fragments according to the attached glycan moiety. In various aspects, the unpaired afucosylated glycans and/or unpaired high mannose glycans of the antibody composition is/are quantified. In various instances, the paired afucosylated glycans and/or paired high mannose glycans of the antibody composition is/are quantified. In various aspects, the unpaired afucosylated glycans, unpaired high mannose glycans, paired afucosylated glycans and the paired high mannose glycans of the antibody composition are quantified. An exemplary method of determining the unpaired glycan content of an antibody composition is described herein at Example 2. Example 5 also demonstrates an exemplary way of determining the unpaired glycan content of an antibody composition.

ADCC and Methods of Modifying ADCC Activity Levels

The data presented herein support that the relative unpaired glycan content of an antibody composition is related to the ADCC activity level for the antibody composition and that the ADCC activity level of the antibody composition may be modified by modifying the relative unpaired glycan content of the antibody composition. Without being bound to a particular theory, the relative unpaired afucosylated glycan content and/or the relative unpaired high mannose glycan content of an antibody composition is related to the ADCC activity level of the antibody composition, and, changing the relative unpaired afucosylated glycan content and/or the relative unpaired high mannose glycan content of the antibody composition leads to changing the ADCC activity level of the antibody composition. It is further contemplated that relative unpaired glycan content (e.g., relative unpaired afucosylated glycan content and/or relative unpaired high mannose glycan content) has greater leverage on ADCC than the relative paired glycan content (e.g., relative paired afucosylated glycan content and/or relative paired high mannose glycan content), so that a percent change in the relative unpaired glycan content will have a greater effect on ADCC than the same percent change in relative paired glycan content. Accordingly, provided herein are methods of modifying the ADCC level of an antibody composition. In exemplary embodiments, the method comprises modifying the relative unpaired afucosylated glycan content of an antibody composition and/or the relative unpaired high mannose glycan content of an antibody composition.

The term “ADCC” or “antibody-dependent cell-mediated cytotoxicity” or “antibody-dependent cellular cytotoxicity” refers to the mechanism by which an effector cell of the immune system (e.g., natural killer cells (NK cells), macrophages, neutrophils, eosinophils) actively lyses a target cell, whose membrane-surface antigens have been bound by specific antibodies. ADCC is a part of the adaptive immune response and occurs when antigen-specific antibodies bind to (1) the membrane-surface antigens on a target cell through its antigen-binding regions and (2) to Fc receptors on the surface of the effector cells through its Fc region. Binding of the Fc region of the antibody to the Fc receptor causes the effector cells to release cytotoxic factors that lead to death of the target cell (e.g., through cell lysis or cellular degranulation).

Fc receptors are receptors on the surfaces of B lymphocytes, follicular dendritic cells, NK cells, macrophages, neutrophils, eosinophils, basophils, platelets and mast cells that bind to the Fc region of an antibody. Fc receptors are grouped into different classes based on the type of antibody that they bind. For example, an Fcγ receptor is a receptor for the Fc region of an IgG antibody, an Fc-alpha receptor is a receptor for the Fc region of an IgA antibody, and an Fc-epsilon receptor is a receptor for the Fc region of an IgE antibody.

The term “FcγR” or “Fc-gamma receptor” is a protein belonging to the IgG superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

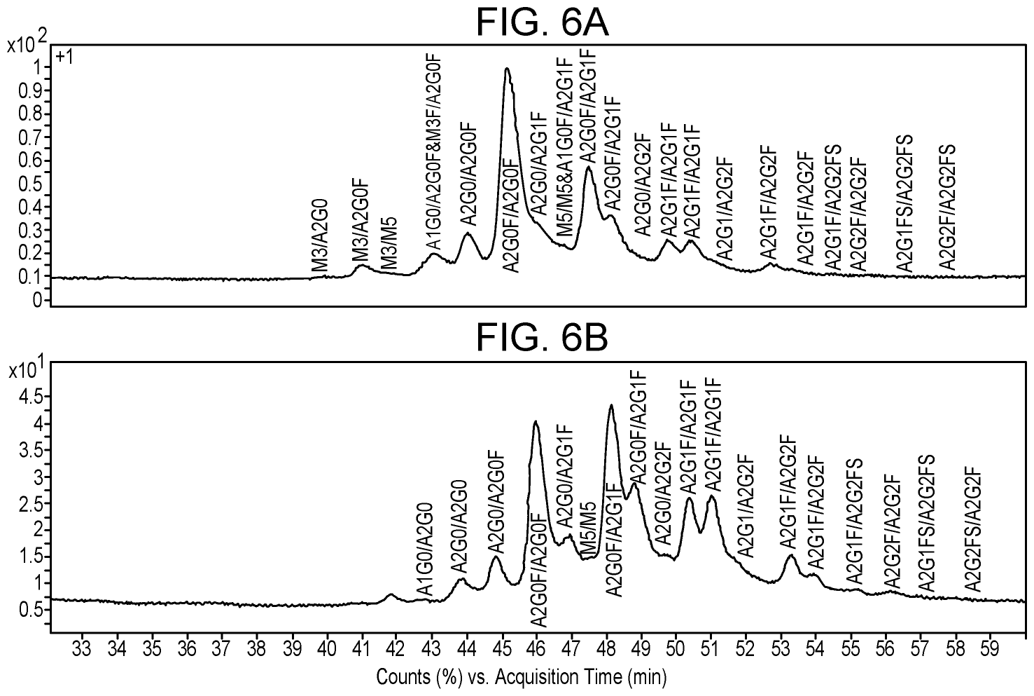

The term “ADCC activity” or “ADCC level” refers to the extent to which ADCC is activated or stimulated. Methods of measuring or determining the ADCC level of an antibody composition, including commercially available assays and kits for measuring or determining the ADCC level, are well-known in the art, as described, Yamashita et al., Scientific Reports 6: article number 19772 (2016), doi:10.1038/srep19772); Kantakamalakul et al., “A novel EGFP-CEM-NKr flow cytometric method for measuring antibody dependent cell mediated-cytotoxicity (ADCC) activity in HIV-1 infected individuals”, J Immunol Methods 315 (Issues 1-2): 1-10; (2006); Gomez-Roman et al., “A simplified method for the rapid fluorometric assessment of antibody-dependent cell-mediated cytotoxicity”, J Immunol Methods 308 (Issues 1-2): 53-67 (2006); Schnueriger et al., Development of a quantitative, cell-line based assay to measure ADCC activity mediated by therapeutic antibodies”, Molec Immunology 38 (Issues 12-13): 1512-1517 (2011); and Mata et al., “Effects of cryopreservation on effector cells for antibody dependent cell-mediated cytotoxicity (ADCC) and natural killer (NK) cell activity in ⁵¹Cr-release and CD107a assays”, J Immunol Methods 406: 1-9 (2014); all herein incorporated by reference for all purposes. The term “ADCC Assay” or “FcγR reporter gene assay” refers to an assay, kit or method useful to determine the ADCC activity of an antibody. Exemplary methods of measuring or determining the ADCC activity of an antibody in the methods described herein include the ADCC assay described in the Example 3 or the ADCC Reporter Assay commercially available from Promega (Catalog No. G7010 and G7018). In some embodiments, ADCC activity is measured or determined using a calcein release assay containing one or more of the following: a FcγRIIIa (158V)-expressing NK92(M1) cells as effector cells and HCC2218 cells or MT-3 cells as target cells labeled with calcein-AM. An illustration of an exemplary calcein release assay is provided as FIG. 8A. In exemplary aspects of the calcein release assay, a standard curve is created using various concentrations of a reference antibody (FIG. 8B).

In exemplary aspects, the level of ADCC of an antibody composition is determined by a quantitative cell-based assay which measures the ability of the antibodies of the antibody composition to mediate cell cytotoxicity in a dose-dependent manner in cells expressing the antigen of the antibodies and engaging FcγRIIIA receptors on effector cells through the Fc domain of the antibodies. In various embodiments, the method comprises the use of target cells harboring detectable labels that are released when the target cells are lysed by the effector cells. The amount of detectable label released from the target cells is a measure of the ADCC activity of the antibody composition. The amount of detectable label released from the target cells, in some aspects, is compared to a baseline. Also, the ADCC level may be reported as a % ADCC relative to a control % ADCC. In various aspects, the % ADCC is a relative % ADCC, which optionally, is relative to a control % ADCC. In various aspects, the control % ADCC is the % ADCC of a reference antibody. In various aspects, the reference antibody is a HER2 antibody (e.g., trastuzumab) or anti-TNF antibody (e.g., infliximab, adalimumab, golimumab, or certolizumab pegol) as described herein. In exemplary instances, the control % ADCC is within a range of about 60% to about 130%. Optionally, the % ADCC is determined by the assay described in Example 3.

In exemplary aspects, the level of ADCC of an antibody composition is determined by measuring the binding between an antibody and an Fc receptor, optionally, FcγRIIIa. In various aspects, the binding activity is a surrogate for ADCC activity since binding of the antibody Fc chain to an Fc receptor is a required event during ADCC. Accordingly, in various instances, ADCC refers to (or is measured as) binding between the antibody and an Fc receptor, such as FcγRIIIa. In various instances, the level of ADCC of an antibody composition is determined by measuring the Fc receptor binding of an antibody using a competitive binding assay wherein the binding of the Fc region of a test antibody is detected by a decrease in fluorescence which represents decreased binding of a reference or control antibody and the Fc receptor. In various aspects, the Fc receptor binding activity is measured as essentially described in Example 4.

In exemplary embodiments, the method of modifying (increasing or decreasing) the ADCC level of an antibody composition comprises modifying (increasing or decreasing) the relative unpaired afucosylated glycan content of an antibody composition and/or the relative unpaired high mannose glycan content of an antibody composition. In exemplary aspects, the presently disclosed method of modifying the ADCC level of an antibody composition comprises increasing the relative unpaired afucosylated glycan content to increase the level of ADCC activity. In exemplary instances, the method of modifying the ADCC level of an antibody composition comprises increasing the relative unpaired high mannose glycan content to increase the level of ADCC activity. In various aspects, the increase in ADCC activity level provided by the methods of the disclosure is at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative to a control. A suitable control may be the same protein or antibody composition without the increase in the relative unpaired glycan content. In exemplary embodiments, the increase in ADCC activity level provided by the methods of the disclosure is about 10% to about 100%, optionally, about 10% to about 90%, about 10% to about 80%, about 10% to about 70%, about 10% to about 70%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%. The increase can be relative to the control. In exemplary embodiments, the increase in ADCC activity level provided by the methods of the disclosure is over 100%, e.g., 200%, 300%, 400%, 500%, 600%, 700%, 800%, 900% or even 1000% relative a control. In exemplary embodiments, the level of ADCC activity increases by at least about 1.5-fold, relative a control. A suitable control may be an ADCC activity level of the same protein or antibody composition without the change in the relative unpaired glycan content. In exemplary embodiments, the level of ADCC activity increases by at least about 2-fold, relative a control. In exemplary embodiments, the level of ADCC activity increases by at least about 3-fold, relative a control. In exemplary embodiments, the level of ADCC activity increases by at least about 4-fold or about 5-fold, relative to a control. In various aspects, the increase in the level of ADCC activity of the antibody composition is related to the increase in relative unpaired glycan content. For instance, the increase in the level of ADCC activity of the antibody composition is at least or about X % per ˜1% increase in relative unpaired glycan content, wherein X % is at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase). Also, for example, X % may be about 10% to about 100%, optionally, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%.

In various aspects, the method of modifying the ADCC level of an antibody composition comprises decreasing the relative unpaired afucosylated glycan content to decrease the level of ADCC activity. In various instances, the method of modifying the ADCC level of an antibody composition comprises decreasing the relative unpaired high mannose glycan content to decrease the level of ADCC activity. In various aspects, the decrease in the ADCC activity level provided by the methods of the disclosure is at least or about a 1% to about a 10% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease) relative a control. A suitable control may be the same protein or antibody composition without the change in the relative unpaired glycan and overall glycan composition content. In exemplary embodiments, the decrease in the ADCC activity level provided by the methods of the disclosure is about 10% to about 100%, optionally, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%. The decrease can be relative to a control. In exemplary embodiments, the decrease in the ADCC activity level provided by the methods of the disclosure is over 100%, e.g., 200%, 300%, 400%, 500%, 600%, 700%, 800%, 900% or even 1000% relative a control. In exemplary embodiments, the level of ADCC activity decreases by at least about 1.5-fold, relative a control. A suitable control may be the ADCC activity level of the same protein or antibody composition without the change in the glycan content. In exemplary embodiments, the level of ADCC activity decreases by at least about 2-fold, relative a control. In exemplary embodiments, the level of ADCC activity decreases by at least about 3-fold, relative a control. In exemplary embodiments, the level of ADCC activity decreases by at least about 4-fold or about 5-fold, relative to a control. In various aspects, the decrease in the level of ADCC activity of the antibody composition is related to the decrease in relative unpaired glycan content. For instance, the decrease in the level of ADCC activity of the antibody composition is at least or about X % per ˜1% decrease in relative unpaired glycan content, wherein X % is at least or about a 1% to about a 10% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease). Also, for example, X % may be about 10% to about 100%, optionally, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%.

In exemplary aspects, the level of ADCC activity is modified by about 10% to about 30% per 1% change in the relative unpaired afucosylated glycan content and/or the relative unpaired high mannose glycan content. For every 1% increase in relative unpaired afucosylated glycan content or relative unpaired high mannose glycan, in some aspects, the level of ADCC activity is increased by about 10% to about 15%. For every 1% decrease in relative unpaired afucosylated glycan content or relative unpaired high mannose glycan, in some aspects, the level of ADCC activity is decreased by about 10% to about 15%. In some aspects, the level of ADCC activity is modified by the sum of about 10% to about 15% times the relative unpaired afucosylated glycan content plus about 10% to about 15% times the relative unpaired high mannose glycan content. Optionally, the level of ADCC activity is modified by the sum of about 12% to about 13% times the relative unpaired afucosylated glycan content plus about 12.5% to about 13.5% times the relative unpaired high mannose glycan content. In exemplary instances, the level of ADCC activity is modified by about 25%, when each of the relative unpaired afucosylated glycan content and the relative unpaired high mannose glycan content is modified by about 1% and the antibody of the antibody composition is an anti-TNF antibody, optionally, infliximab, adalimumab, golimumab, or certolizumab pegol. In exemplary instances, the level of ADCC activity is increased by about 20% to about 30%, when each of the relative unpaired afucosylated glycan content and the relative unpaired high mannose glycan content is increased by about 1% and the antibody of the antibody composition is an anti-TNF antibody, optionally, infliximab, adalimumab, golimumab, or certolizumab pegol. In exemplary instances, the level of ADCC activity is decreased by about 20% to about 30%, when each of the relative unpaired afucosylated glycan content and the relative unpaired high mannose glycan content is decreased by about 1% and the antibody of the antibody composition is an anti-TNF antibody, optionally, infliximab, adalimumab, golimumab, or certolizumab pegol.

In exemplary aspects, the level of ADCC activity is modified by about 10% to about 20% per 1% change in the relative unpaired afucosylated glycan content. For every 1% increase in relative unpaired afucosylated glycan content, in some aspects, the level of ADCC activity is increased by about 10% to about 20%. For every 1% decrease in relative unpaired afucosylated glycan content or relative unpaired high mannose glycan, in some aspects, the level of ADCC activity is decreased by about 10% to about 20%. In various instances, the ADCC activity is unchanged per 1% change in the relative unpaired high mannose glycan content. Optionally, the level of ADCC activity is modified by about 10% to about 19%, about 10% to about 18%, about 10% to about 17%, about 10% to about 16%, about 10% to about 15%, about 11% to about 20%, about 12% to about 20%, about 13% to about 20%, about 12% to about 16%, e.g., about 13%, about 14%, about 15%, per about 1% change in the relative unpaired afucosylated glycan content. Optionally, the antibody of the antibody composition is an anti-HER2 antibody, optionally, trastuzumab or pertuzumab.

In exemplary aspects, the modification (increase or decrease) effected by the presently disclosed methods are relative to a “control”. In exemplary aspects, the control is the level of ADCC activity when the steps of the method are not carried out. In exemplary aspects, the control is the level of ADCC activity when the relative unpaired afucosylated glycan content and/or the relative unpaired high mannose glycan content is not modified (increased or decreased). For example, a suitable control may be the ADCC activity level of the same protein or antibody composition but without the increase in the relative unpaired glycan content (e.g., relative unpaired afucosylated glycan content and/or the relative unpaired high mannose glycan content), or a suitable control may be the ADCC activity level of the same protein or antibody composition but without the decrease in the relative unpaired glycan content (e.g., relative unpaired afucosylated glycan content and/or the relative unpaired high mannose glycan content). In exemplary instances, the control may be the ADCC activity level of the same protein or antibody composition produced under the same cell culture conditions with exception of those conditions that were modified to cause a change in the relative unpaired glycan content. In exemplary aspects, the control may be the ADCC activity level of the same protein or antibody composition produced under a first set of cell culture conditions which lead to an ADCC activity level which is outside of a target range of ADCC activity level. In various aspects, the control may be the ADCC activity level of the same protein or antibody composition produced under a first set of cell culture conditions which lead to a relative unpaired afucosylated glycan content and/or the relative unpaired high mannose glycan content which is/are outside of a target range.

Methods of Modifying Unpaired Glycan Content

In exemplary embodiments, the method of modifying (increasing or decreasing) the ADCC level of an antibody composition comprises modifying (increasing or decreasing) the unpaired afucosylated glycan content of an antibody composition and/or the unpaired high mannose glycan content of an antibody composition. In exemplary aspects, the presently disclosed method of modifying the ADCC level of an antibody composition comprises increasing the unpaired afucosylated glycan content and/or the unpaired high mannose content to increase the level of ADCC activity. In various aspects, the method of modifying the ADCC level of an antibody composition comprises increasing the unpaired afucosylated glycan content and/or the unpaired high mannose content by at least about 1%, at least about 2%, at least about 3%, at least about 4%, at least about 5%, or more. In various aspects, the method of modifying the ADCC level of an antibody composition comprises increasing the unpaired afucosylated glycan content and/or the unpaired high mannose content by more than 5% or more than 10%, e.g., by about 5%, about 6%, about 7%, about 8%, about 9%, about 10%, about 11%, about 12%, about 13%, about 14%, about 15%, about 16%, about 17%, about 18%, about 19%, or about 20%. In various aspects, the method comprises increasing the unpaired afucosylated glycan content and/or the unpaired high mannose content by at least about 25%, at least about 30%, at least about 40%, at least about 50%, at least about 60%, at least about 70%, at least about 80%, at least about 90%, or more.

In exemplary aspects, the presently disclosed method of modifying the ADCC level of an antibody composition comprises decreasing the unpaired afucosylated glycan content to decrease the level of ADCC activity. In various aspects, the method of modifying the ADCC level of an antibody composition comprises decreasing the unpaired afucosylated glycan content and/or the unpaired high mannose content by at least about 1%, at least about 2%, at least about 3%, at least about 4%, at least about 5%, or more. In various aspects, the method of modifying the ADCC level of an antibody composition comprises decreasing the unpaired afucosylated glycan content and/or the unpaired high mannose content by more than 5% or more than 10%, e.g., by about 5%, about 6%, about 7%, about 8%, about 9%, about 10%, about 11%, about 12%, about 13%, about 14%, about 15%, about 16%, about 17%, about 18%, about 19%, or about 20%. In various aspects, the method comprises decreasing the unpaired afucosylated glycan content and/or the unpaired high mannose content by at least about 25%, at least about 30%, at least about 40%, at least about 50%, at least about 60%, at least about 70%, at least about 80%, at least about 90%, or more.

In exemplary aspects, the increase or decrease in the unpaired afucosylated glycan content and/or unpaired high mannose glycan content is/are relative to a “control”. In exemplary aspects, the control is the unpaired glycan content of a control protein or antibody composition produced under the same cell culture conditions with exception of those conditions that lead to increased or decreased unpaired glycan content. In exemplary aspects, the control may be the unpaired glycan content of the same protein or antibody composition produced under a first set of cell culture conditions which lead to an ADCC activity level which is outside of a target range of ADCC activity level. In various aspects, the control may be the unpaired glycan content of the same protein or antibody composition produced under a first set of cell culture conditions which lead to an unpaired afucosylated glycan content and/or the unpaired high mannose glycan content which is/are outside of a target range.

Without being bound to a particular theory, conditions which lead to a modified (increased or decreased) afucosylated glycan content and/or high mannose content may lead to increased or decreased unpaired afucosylated glycan content and/or unpaired high mannose content of an antibody composition. In various instances, the unpaired afucosylated glycan content and/or unpaired high mannose content is increased or decreased by following the teachings of any one of International Patent Application Publication Nos. WO2013/114164, WO2013/114245, WO2013/114167, WO2015128793, or WO2016/089919, WO2018/170099, WO2019/191150, each of which is incorporated herein by reference. In various instances, the unpaired afucosylated glycan content and/or unpaired high mannose content is increased or decreased by selecting a clone that produces antibody or antibody protein product comprising a specified level of unpaired afucosylated glycan content and/or unpaired high mannose content.

Methods of Producing Antibody Compositions

Simple and efficient methods to predict the level of effector function (e.g., ADCC) a particular antibody composition will exhibit based on a given glycoform profile for that antibody composition are described herein. The data provided herein support that the ADCC activity level for the antibody composition may be predicted based on the relative unpaired glycan content of an antibody composition. Without being bound to a particular theory, the unpaired afucosylated glycan content and/or the relative unpaired high mannose glycan content of an antibody composition is predictive of the ADCC activity level of the antibody composition. Such predicted ADCC levels are useful during antibody production, when, it is necessary for the antibody to have an ADCC activity level within a target range. For instance, by monitoring the unpaired afucosylated glycan content and/or the unpaired high mannose glycan content of an antibody composition, it may be predicted whether the antibody composition will exhibit an ADCC activity level within a target range. If a target range of ADCC activity levels for an antibody composition is known, the target range of relative unpaired afucosylated glycan content and/or the relative unpaired high mannose glycan content may be determined. Selection of the antibody composition for continued processing, e.g., downstream processing, may occur when the unpaired afucosylated glycan content and/or the unpaired high mannose glycan, or the ADCC activity level, as calculated based on the unpaired afucosylated glycan content and/or the unpaired high mannose glycan content, is/are within a target range. In exemplary aspects, the target range is based on a target range of ADCC activity levels for a reference antibody and a model which correlates ADCC activity level of the antibody composition to afucosylated glycan content and/or high mannose glycan content of the antibody composition, optionally, a model which correlates ADCC activity level of the antibody composition to unpaired afucosylated glycan content and/or unpaired high mannose glycan content of the antibody composition. In exemplary instances, the reference antibody is infliximab. In exemplary aspects, the reference antibody is trastuzumab. Optionally, the target range of the unpaired afucosylated glycan content is about 80% to about 90% and/or the target range of the unpaired high mannose glycan is about 75% to about 85%, when the antibody is an anti-TNF antibody, such as, infliximab. Optionally, the target range of the unpaired afucosylated glycan content is about 95% to about 99% and/or the target range of the unpaired high mannose glycan is below about 25% to about 35%, when the antibody is an anti-HER2 antibody, such as, trastuzumab.

Accordingly, the present disclosure provides methods of producing an antibody composition. In exemplary embodiments, the method comprises (i) determining the relative unpaired afucosylated glycan content and/or the relative unpaired high mannose glycan content of a sample of the antibody composition; and (ii) selecting the antibody composition for downstream processing based on the relative unpaired afucosylated glycan content and/or relative unpaired high mannose glycan content determined in (i). In exemplary embodiments, the method of producing an antibody composition comprises (i) determining the unpaired afucosylated glycan content of an antibody composition and/or the unpaired high mannose glycan content of an antibody composition; (ii) determining the ADCC level of the antibody composition based on the unpaired afucosylated glycan content and/or the unpaired high mannose glycan content determined in (i); and (iii) selecting the antibody composition for downstream processing when the ADCC level of the antibody composition determined in (ii) is within a target ADCC range. In exemplary embodiments, the method of producing an antibody composition comprises (i) determining the unpaired afucosylated glycan content and/or the unpaired high mannose glycan content of a sample of the antibody composition taken from a cell culture comprising glycosylation-competent cells expressing an antibody of the antibody composition; (ii) optionally, modifying the cell culture to modulate the unpaired afucosylated glycan content and/or the unpaired high mannose glycan content and determining the unpaired afucosylated glycan content and/or the unpaired high mannose glycan content of a sample of the antibody composition taken from the modified cell culture; and (iii) selecting the antibody composition for downstream processing based on the unpaired afucosylated glycan content and/or unpaired high mannose glycan content.

In various aspects, the sample is taken from a cell culture comprising glycosylation-competent cells expressing an antibody of the antibody composition. Optionally, the method further comprises modifying one or more conditions of the cell culture to modify the relative unpaired afucosylated glycan content and/or the relative unpaired high mannose glycan content of the antibody composition and determining the relative unpaired afucosylated glycan content and/or the relative unpaired high mannose glycan content of a sample of the antibody composition taken from the modified cell culture. In exemplary aspects, the method comprises repeating the modifying until the relative unpaired afucosylated glycan content and/or relative unpaired high mannose glycan content is within a target range. In various aspects, the relative unpaired afucosylated glycan content and/or the relative unpaired high mannose glycan content is/are determined in real time with respect to production of the antibody composition. As used herein “real time” refers to determinations that are made while a production process is ongoing, without interruption to the process. It will be appreciated that production of therapeutic proteins involves living cells and sensitive materials that cannot be put on hold indefinitely while assays and determinations are performed. If numerical examples of interest, a “real time” determination can be a determination that is made within 6 hours, 5 hours, 4 hours, 3 hours, 2 hours, 1 hour, minutes, 40 minutes, 30 minutes, 20 minute, 10 minutes, 5 minutes, 1 minute, 30 seconds, seconds, 10 seconds, 5 seconds, 1 seconds, or 0.1 seconds from the time a measurement is made (while the production process is ongoing). In various instances, the method comprises selecting the antibody composition for downstream processing when the relative unpaired afucosylated glycan content and/or relative unpaired high mannose glycan content is/are in a target range. In various aspects, the relative unpaired afucosylated glycan content and/or relative unpaired high mannose glycan content correlate with the ADCC activity level of the antibody composition. Optionally, the method further comprises determining the ADCC activity level of the antibody composition based on the relative unpaired afucosylated glycan content and/or relative unpaired high mannose glycan content determined in (i). Optionally, the method comprises selecting the antibody composition for downstream processing when the ADCC activity level is in a target range.

In various aspects, the method of producing an antibody composition comprises modifying the ADCC level of an antibody composition according to a method of modifying the ADCC level of the present disclosure.

In various instances, the method of producing an antibody composition comprises determining the relative unpaired glycan content of an antibody composition according to any of the presently disclosed methods of determining the relative unpaired glycan content of an antibody composition. For example, the determining comprises (i) treating the antibody composition with an enzyme which cleaves an antibody heavy chain at a site N-terminal to the hinge region disulfide linkages to form antibody fragments, (ii) separating the antibody fragments by a chromatography, and (iii) quantifying each antibody fragment. In various instances, the site is between Thr and His or between Lys and Thr of the sequence KTHTCPP (SEQ ID NO: 1) of an IgG1 antibody heavy chain. In various aspects, the antibody fragments comprise Fab fragments and glycosylated Fc fragments. In exemplary aspects, the separating of (ii) comprises hydrophilic interaction liquid chromatography (HILIC). In exemplary aspects, the quantifying of (iii) comprises mass spectrometry. In various aspects, the determining comprises the method (or portions thereof) of determining the relative unpaired glycan content of an antibody composition described herein at Example 2.

Downstream Processing

The relative % unpaired high mannose glycans and/or relative % unpaired afucosylated glycans are determined (e.g., measured) to better inform as to the % antibody-dependent cell-mediated cytotoxicity (ADCC) of the antibody composition. The determining (e.g., measuring) may occur at any stage of manufacture. In particular, measurements may be taken pre- or post-harvest, preceding or during any stage of downstream processing. Example downstream processing includes any chromatography unit operation, including capture chromatography, intermediate chromatography, and/or polish chromatography unit operations; virus inactivation and neutralization; virus filtration; and/or final formulation. The relative % unpaired high mannose glycans, and/or relative % unpaired afucosylated glycans in various aspects is determined (e.g., measured) in real-time, near real-time, and/or after the fact. Monitoring and measurements can be done using known techniques and commercially available equipment.

In various aspects of the present disclosure, the determining (e.g., measuring) the % unpaired high mannose glycans and/or % unpaired afucosylated glycans is carried out before a harvest. As used herein the term “harvest” refers to cell culture media containing the recombinant protein of interest being collected and separated at least from the cells of the cell culture. The harvest can be performed continuously. The harvest in some aspects is performed using centrifugation and can further comprise precipitation, filtration, and the like. In various aspects, the determining is carried out before harvest. In various aspects, the determining is carried out before chromatography, optionally, Protein A chromatography. In some aspects, the determining (e.g., measuring) the relative % unpaired high mannose glycans and/or relative % unpaired afucosylated glycans is carried out at least 3 days, at least 4 days, or at least 5 days before harvest. Optionally, determining (e.g., measuring) the relative % unpaired high mannose glycans and/or relative % unpaired afucosylated glycans is carried out in real-time with regard to antibody production.

In various aspects of the present disclosure, determining (e.g., measuring) the relative % unpaired high mannose glycans, and/or relative % unpaired afucosylated glycans is carried out after a harvest. In various aspects, the determining is carried out after chromatography, optionally, Protein A chromatography. In various aspects, the determining is carried out after harvest and after chromatography, e.g., a Protein A chromatography.

With regard to the presently disclosed methods, the antibody composition in various aspects is selected or chosen for further processing, e.g., for downstream processing, and the selection is based on a particular parameter, e.g., % ADCC, relative % unpaired high mannose glycans, and/or relative % unpaired afucosylated glycans. In various instances, the presently disclosed methods comprise using the antibody composition in further processing, e.g., downstream processing, based on a particular parameter, e.g., based on the % ADCC, relative % unpaired high mannose glycans, and/or relative % unpaired afucosylated glycans. In various instances, the presently disclosed methods comprise carrying out further processing, e.g., downstream processing, with the antibody composition, based on a particular parameter, e.g., based on the % ADCC, relative % unpaired high mannose glycans, and/or relative % unpaired afucosylated glycans.

In exemplary instances the downstream processing comprises or consists of any processing which occurs after (or downstream of) the processing at which the relative % unpaired high mannose glycans, and/or relative % unpaired afucosylated glycans are determined (e.g., measured). For example, if the relative % unpaired high mannose glycans, and/or relative % unpaired afucosylated glycans were determined (e.g., measured) at harvest, then the downstream processing is any processing which occurs after (or downstream of) the harvest, which in various aspects comprise(s): dilution, filling, filtration, formulation, chromatography, viral filtration, viral inactivation, or a combination thereof. Also, for example, if the relative % unpaired high mannose glycans, and/or relative % unpaired afucosylated glycans were determined (e.g., measured) after chromatography, e.g., Protein A chromatography, then the downstream processing comprises or consists of any processing which occurs after (or downstream of) the chromatography, and the downstream processing in various aspects comprise(s): a dilution, a filling, a filtration, a formulation, further chromatography, a viral filtration, a viral inactivation, or a combination thereof. In exemplary instances the further chromatography is an ion exchange chromatography (e.g., cation exchange chromatography or anion exchange chromatography).

Stages/types of chromatography used during downstream processing include capture or affinity chromatography which is used to separate the recombinant product from other proteins, aggregates, DNA, viruses and other such impurities. In exemplary instances, initial chromatography is carried out with Protein A (e.g., Protein A attached to a resin). Intermediate and polish chromatography in various aspects further purify the recombinant protein, removing bulk contaminants, adventitious viruses, trace impurities, aggregates, isoforms, etc. The chromatography can either be performed in bind and elute mode, where the recombinant protein of interest is bound to the chromatography medium and the impurities flow through, or in flow-through mode, where the impurities are bound and the recombinant protein flows through. Examples of such chromatography methods include ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography and gel filtration.

In various aspects, the downstream processing comprises viral inactivation. Enveloped viruses have a capsid enclosed by a lipoprotein membrane or “envelope” and are therefore susceptible to inactivation. The virus inactivation in various instances includes heat inactivation/pasteurization, pH inactivation, UV and gamma ray irradiation, use of high intensity broad spectrum white light, addition of chemical inactivating agents, surfactants, and solvent/detergent treatments.

In various aspects, the downstream processing comprises virus filtration. In various aspects, the virus filtration comprises removing non-enveloped viruses. In various aspects, the virus filtration comprises the use of micro- or nano-filters.

In various aspects, the downstream processing comprises formulation, which may be performed in one or more steps. Following completion of the chromatography, the purified recombinant proteins are in various aspects buffer exchanged into a formulation buffer. In exemplary aspects, the buffer exchange is performed using ultrafiltration and diafiltration (UF/DF). In exemplary aspects, the recombinant protein is buffer exchanged into a desired formulation buffer using diafiltration and concentrated to a desired final formulation concentration using ultrafiltration. Additional stability-enhancing excipients in various aspects are added following a UF/DF formulation.

Recombinant Glycosylated Proteins

The presently disclosed methods relate to compositions comprising a recombinant glycosylated protein. In various aspects, the recombinant glycosylated protein comprises an amino acid sequence comprising one or more N-glycosylation consensus sequences of the formula:

Asn-Xaa₁-Xaa₂

wherein Xaa₁ is any amino acid except Pro, and Xaa₂ is Ser or Thr.

In exemplary embodiments, the recombinant glycosylated protein comprises a fragment crystallizable (Fc) polypeptide. The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. In exemplary embodiments, the recombinant glycosylated protein comprises the Fc of an IgG, e.g., a human IgG. In exemplary aspects, the recombinant glycosylated protein comprises the Fc an IgG1 or IgG2. In exemplary aspects, the recombinant glycosylated protein is an antibody, an antibody protein product, a peptibody, or a Fc-fusion protein.

In exemplary aspects, the recombinant glycosylated protein is an antibody. As used herein, the term “antibody” has its customary and ordinary meaning as understood by one of ordinary skill in the art in view of this disclosure. It refers to a protein having a canonical immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody canonically comprises a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. Thus, the antibody may comprise a heavy chain comprising a variable region comprising three CDRS, and a light chain comprising a variable region comprising three CDRs. See, e.g., Janeway et al., “Structure of the Antibody Molecule and the Immunoglobulin Genes”, Immunobiology: The Immune System in Health and Disease, 4^{th} ed. Elsevier Science Ltd./Garland Publishing, (1999).

Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

In various aspects, the antibody can be a monoclonal antibody or a polyclonal antibody. In exemplary instances, the antibody is a mammalian antibody, e.g., a mouse antibody, rat antibody, rabbit antibody, goat antibody, horse antibody, chicken antibody, hamster antibody, pig antibody, human antibody, and the like. In certain aspects, the recombinant glycosylated protein is a monoclonal human antibody.

An antibody, in various aspects, is cleaved into fragments by enzymes, such as, e.g., papain, pepsin, and/or gingipain K. Papain cleaves an antibody to produce two Fab fragments and a single Fc fragment. Pepsin cleaves an antibody to produce a F(ab′)₂ fragment and a pFc′ fragment. In exemplary aspects, the recombinant glycosylated protein is an antibody fragment, e.g., a Fab, Fc, F(ab′)₂, or a pFc′, that retains at least one glycosylation site. With regard to the methods of the disclosure, the antibody may lack certain portions of an antibody, and may be an antibody fragment. In various aspects, the antibody fragment comprises a glycosylation site. In some aspects, the fragment is a “Glycosylated Fc Fragment” which comprises at least a portion of the Fc region of an antibody which is glycosylated post-translationally in eukaryotic cells. In various instances, the recombinant glycosylated protein is glycosylated Fc fragment.

The architecture of antibodies has been exploited to create a growing range of alternative formats that spans a molecular-weight range of at least or about 12-150 kDa and a valency (n) range from monomeric (n=1), dimeric (n=2) and trimeric (n=3) to tetrameric (n=4) and potentially higher; such alternative antibody formats are referred to herein as “antibody protein products” or “binding proteins”.

Antibody protein products can be an antigen binding format based on antibody fragments, e.g., scFvs, Fabs and VHH/VH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the recombinant glycosylated protein comprises any one of these antibody protein products (e.g., scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate) and comprises one or more N-glycosylation consensus sequences, optionally, one or more Fc polypeptides. In various aspects, the antibody protein product comprises a glycosylation site. In exemplary aspects, an antibody protein product can be a Glycosylated Fc Fragment conjugated to an antibody binding fragment (“Glycosylated Fc Fragment antibody product”).

The recombinant glycosylated protein may be an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In various aspects, the recombinant glycosylated protein is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

Advantageously, the methods are not limited to the antigen-specificity of the antibody, glycosylated Fc fragment, antibody protein product, chimeric antibody, or humanized antibody. Accordingly, the antibody, glycosylated Fc fragment, antibody protein product, chimeric antibody, or humanized antibody has any binding specificity for virtually any antigen. In exemplary aspects, the antibody binds to a hormone, growth factor, cytokine, a cell-surface receptor, or any ligand thereof. In exemplary aspects, the antibody binds to a protein expressed on the cell surface of an immune cell. In exemplary aspects, the antibody binds to a cluster of differentiation molecule selected from the group consisting of: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD798, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the antibody, glycosylated Fc fragment, antibody protein product, chimeric antibody, or humanized antibody is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-8 type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor I), U.S. Pat. Nos. 7,566,772 and 7,964,193 (interleukin-1β), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publication No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-8 type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5) U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antibody protein product or antibody.

In exemplary embodiments, the antibody, glycosylated Fc fragment, antibody protein product, chimeric antibody, or humanized antibody is one of Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), I'Ibritumomab tiuxetan (product marketed with the brand name Zevalin®), I'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), I'Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody, glycosylated Fc fragment, antibody protein product, chimeric antibody, or humanized antibody is one of anti-TNF alpha proteins such as adalimumab, infliximab, etanercept, golimumab, and certolizumab pegol; anti-TNF alpha antibodies such as adalimumab, infliximab, golimumab, and certolizumab pegol; anti-IL1beta antibodies such as canakinumab; anti-IL12/23 (p40) antibodies such as ustekinumab and briakinumab; and anti-IL2R antibodies, such as daclizumab. It will be understood that nonproprietary names of molecules described herein will include any innovator or biosimilar product comprising the molecule of that nonproprietary name. In exemplary embodiments, the antibody, glycosylated Fc fragment, antibody protein product, chimeric antibody, or humanized antibody is a biosimilar of one of the aforementioned antibodies.

In exemplary aspects, the antibody binds to a tumor associated antigen and is an anti-cancer antibody. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies, such as siltuximab; and anti-VEGF antibodies such as bevacizumab; anti-IL6 receptor antibodies such as tocilizumab.

In exemplary aspects, the antigen of the antibody is TNFα and the antibody is an anti-TNFα antibody (which may also be referred to as simply an “anti-TNF” antibody for conciseness), e.g., an anti-TNFα monoclonal antibody. In exemplary aspects, the antigen of the antibody comprises SEQ ID NO: 2. In various aspects, the antibody is infliximab. The term infliximab refers to a chimeric, monoclonal IgG1 kappa antibody composed of human constant and murine variable regions and binds TNFα antigen (See CAS Number: 170277-31-3, DrugBank Accession No. DB00065). Infliximab, also known as chimeric antibody cA2, was derived from a murine monoclonal antibody called A2 (Knight et al., Molec Immunol 30(16): 1443-1453 (1993)). The variable region of the cA2 light chain and of the cA2 light chain are published in International Publication No. WO 2006/065975. In exemplary aspects, the antibody comprises a light chain comprising a CDR1, CDR2, and CDR3 of the variable region of the infliximab light chain as set forth in Table A. In exemplary aspects, the antibody comprises a heavy chain comprising a CDR1, CDR2, and CDR3 of the variable region of the infliximab heavy chain as set forth in Table A. In various instances, the antibody comprises the VH and VL or comprising VH-IgG1 and VL-IgG kappa sequences of infliximab.

TABLE A
Infliximab Amino Acid Sequences

| Description | Sequence | SEQ ID NO: |
|---|---|---|
| VL | DILLTQSPAILSVSPGERVSFSCRASQFVGSSIHWYQQRTNGSPRLLIKYASESMSGIPSRF | 3 |
| | SGSGSGTDFTLSINTVESEDIADYYCQQSHSWPFTFGSGTNLEVK | |
| VH | EVKLEESGGGLVQPGGSMKLSCVASGFIFSNHWMNWVRQSPEKGLEWVAEIRSKSINS | 4 |
| | ATHYAESVKGRFTISRDDSKSAVYLQMTDLRTEDTGVYYCSRNYYGSTYDYWGQGTTLT | |
| | VS | |
| Full length | DILLTQSPAI LSVSPGERVS FSCRASQFVG SSIHWYQQRT NGSPRLLIKY ASESMSGIPS | 5 |
| LC | RFSGSGSGTD FTLSINTVES EDIADYYCQQ SHSWPFTFGS GTNLEVKRTV | |
| | AAPSVFIFPP SDEQLKSGTA SVVCLLNNFY PREAKVQWKV DNALQSGNSQ | |
| | ESVTEQDSKD STYSLSSTLT LSKADYEKHK VYACEVTHQG LSSPVTKSEN RGEC | |
| Full length | EVKLEESGGG LVQPGGSMKL SCVASGFIFS NHWMNWVRQS PEKGLEWVAE | 6 |
| HC | IRSKSINSAT HYAESVKGRF TISRDDSKSA VYLQMTDLRT EDTGVYYCSR | |
| | NYYGSTYDYW GQGTTLTVSS ASTKGPSVFP LAPSSKSTSG GTAALGCLVK | |
| | DYFPEPVTVS WNSGALTSGV HTFPAVLQSS GLYSLSSVVT VPSSSLGTQT | |
| | YICNVNHKPS NTKVDKKVEP KSCDKTHTCP PCPAPELLGG PSVFLFPPKP | |
| | KDTLMISRTP EVTCVVVDVS HEDPEVKFNW YVDGVEVHNA KTKPREEQYN | |
| | STYRVVSVLT VLHQDWLNGK EYKCKVSNKA LPAPIEKTIS KAKGQPREPQ | |
| | VYTLPPSRDE LTKNQVSLTC LVKGFYPSDI AVEWESNGQP ENNYKTTPPV | |
| | LDSDGSFFLY SKLTVDKSRW QQGNVFSCSV MHEALHNHYT QKSLSLSPGK | |

LC, light chain; HC, heavy chain; VL, variable light chain; VH, variable heavy chain.

In various aspects, the antibody comprises:
i. a CDR1 of the light chain (LC) variable region of SEQ ID NO: 3; or an amino acid sequence which is at least 90% (e.g., at least 95%, at least 96%, at least 97%, at least 98% or at least 99%) identical to the LC CDR1 amino acid sequence; or a variant amino acid sequence of the LC CDR1 amino acid sequence with 1 or 2 amino acid substitutions,
ii. a CDR2 of the LC variable region of SEQ ID NO: 3; or an amino acid sequence which is at least 90% (e.g., at least 95%, at least 96%, at least 97%, at least 98% or at least 99%) identical to the LC CDR2 amino acid sequence; or a variant amino acid sequence of the LC CDR2 amino acid sequence with 1 or 2 amino acid substitutions,
iii. a CDR3 of the LC variable region of SEQ ID NO: 3; or an amino acid sequence which is at least 90% (e.g., at least 95%, at least 96%, at least 97%, at least 98% or at least 99%) identical to the LC CDR3 amino acid sequence; or a variant amino acid sequence of the LC CDR3 amino acid sequence with 1 or 2 amino acid substitutions,
iv. a CDR1 of the heavy chain (HC) variable region of SEQ ID NO: 4; or an amino acid sequence which is at least 90% (e.g., at least 95%, at least 96%, at least 97%, at least 98% or at least 99%) identical to the HC CDR1 amino acid sequence; or a variant amino acid sequence of the HC CDR1 amino acid sequence with 1 or 2 amino acid substitutions;
v. a CDR2 of the heavy chain (HC) variable region of SEQ ID NO: 4; or an amino acid sequence which is at least 90% (e.g., at least 95%, at least 96%, at least 97%, at least 98% or at least 99%) identical to the HC CDR2 amino acid sequence; or a variant amino acid sequence of the HC CDR2 amino acid sequence with 1 or 2 amino acid substitutions; and
vi. a CDR3 of the heavy chain (HC) variable region of SEQ ID NO: 4; or an amino acid sequence which is at least 90% (e.g., at least 95%, at least 96%, at least 97%, at least 98% or at least 99%) identical to the HC CDR3 amino acid sequence; or a variant amino acid sequence of the HC CDR3 amino acid sequence with 1 or 2 amino acid substitutions.

In various instances, the antibody comprises: a LC variable region comprising an amino acid sequence of SEQ ID NO: 3, an amino acid sequence which is at least 90% (e.g., at least 95%, at least 96%, at least 97%, at least 98% or at least 99%) identical to SEQ ID NO: 3, or a variant amino acid sequence of SEQ ID NO: 3 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the antibody comprises: a HC variable region comprising an amino acid sequence of SEQ ID NO: 4, an amino acid sequence which is at least 90% (e.g., at least 95%, at least 96%, at least 97%, at least 98% or at least 99%) identical to SEQ ID NO: 4, or a variant amino acid sequence of SEQ ID NO: 4 with 1 to 10 (e.g., 1 to 9, 1 to 8, 1 to 7, 1 to 6, 1 to 5, 1 to 4, 1 to 3, 1 or 2) amino acid substitutions.

In exemplary instances, the antibody comprises a light chain comprising an amino acid sequence of SEQ ID NO: 5, an amino acid sequence which is at least 90% (e.g., at least 95%, at least 96%, at least 97%, at least 98% or at least 99%) identical to SEQ ID NO: 5, or a variant amino acid sequence of SEQ ID NO: 5 with 1 to 10 (e.g., 1 to 9, 1 to 8, 1 to 7, 1 to 6, 1 to 1 to 4, 1 to 3, 1 or 2) amino acid substitutions.

In various aspects, the antibody comprises a heavy chain comprising an amino acid sequence of SEQ ID NO: 6, an amino acid sequence which is at least 90% (e.g., at least 95%, at least 96%, at least 97%, at least 98% or at least 99%) identical to SEQ ID NO: 6, or a variant amino acid sequence of SEQ ID NO: 6 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the tumor associated antigen is HER2 and the antibody is an anti-HER2 antibody, e.g., an anti-HER2 monoclonal antibody. In exemplary aspects, the tumor associated antigen comprises SEQ ID NO: 7. In various aspects, the IgG1 antibody is trastuzumab. The term trastuzumab refers to an IgG1 kappa, humanized, monoclonal antibody that binds HER2 antigen (see CAS Number: 180288-69-1, DrugBank Accession No. DB00072). In exemplary aspects, the antibody comprises a light chain comprising a CDR1, CDR2, and CDR3 as set forth in Table B. In exemplary aspects, the antibody comprises a heavy chain comprising a CDR1, CDR2, and CDR3 as set forth in Table B. In various instances, the antibody comprises the VH and VL or comprising VH-IgG1 and VL-IgG kappa sequences recited in Table B.

TABLE B
Trastuzumab Amino Acid Sequences

| Description | Sequence | SEQ ID NO: |
|---|---|---|
| LC CDR1 | QDVNTA | 8 |
| LC CDR2 | SAS | 9 |
| LC CDR3 | QQHYTTPPT | 10 |
| HC CDR1 | GFNIKDTY | 11 |
| HC CDR2 | IYPTNGYT | 12 |
| HC CDR3 | SRWGGDGFYAMDY | 13 |
| VL | DIQMTQSPSSLSASVGDRVTITCRASQDVNTAVAWYQQKPGKAPKLLIYSASFLYSGV | 14 |
| | PSRFSGSRSGTDFTLTISSLQPEDFATYYCQQHYTTPPTFGQGTKVEIK | |
| VH | EVQLVESGGGLVQPGGSLRLSCAASGFNIKDTYIHWVRQAPGKGLEWVARIYPTNGY | 15 |
| | TRYADSVKGRFTISADTSKNTAYLQMNSLRAEDTAVYYCSRWGGDGFYAMDYWGQ | |
| | GTLVTVSS | |
| Full length | DIQMTQSPSSLSASVGDRVTITCRASQDVNTAVAWYQQKPGKAPKLLIYSASFLYSGV | 16 |
| LC | PSRFSGSRSGTDFTLTISSLQPEDFATYYCQQHYTTPPTFGQGTKVEIKRTVAAPSVFIFP | |
| | PSDEQLKSGTASVVCLLNNFYPREAKVQWKVDNALQSGNSQESVTEQDSKDSTYSLS | |
| | STLTLSKADYEKHKVYACEVTHQGLSSPVTKSFNRGEC | |
| Full length | EVQLVESGGGLVQPGGSLRLSCAASGFNIKDTYIHWVRQAPGKGLEWVARIYPTNGY | 17 |
| HC | TRYADSVKGRFTISADTSKNTAYLQMNSLRAEDTAVYYCSRWGGDGFYAMDYWGQ | |
| | GTLVTVSSASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPPKSCDKTHTCP | |
| | PCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQP | |
| | REPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |

LC, light chain; HC, heavy chain; VL, variable light chain; VH, variable heavy chain.

In various aspects, the antibody comprises:
i. a light chain (LC) CDR1 comprising an amino acid sequence of SEQ ID NO: 8 or an amino acid sequence which is at least 90% (e.g., at least 95%, at least 96%, at least 97%, at least 98% or at least 99%) identical to SEQ ID NO: 8 or a variant amino acid sequence of SEQ ID NO: 8 with 1 or 2 amino acid substitutions,
ii. a LC CDR2 comprising an amino acid sequence of SEQ ID NO: 9 or an amino acid sequence which is at least 90% (e.g., at least 95%, at least 96%, at least 97%, at least 98% or at least 99%) identical to SEQ ID NO: 9 or a variant amino acid sequence of SEQ ID NO: 9 with 1 or 2 amino acid substitutions,
iii. a LC CDR3 comprising an amino acid sequence of SEQ ID NO: 10 or an amino acid sequence which is at least 90% (e.g., at least 95%, at least 96%, at least 97%, at least 98% or at least 99%) identical to SEQ ID NO: 10 or a variant amino acid sequence of SEQ ID NO: 10 with 1 or 2 amino acid substitutions,
iv. a heavy chain (HC) CDR1 comprising an amino acid sequence of SEQ ID NO: 11 or an amino acid sequence which is at least 90% (e.g., at least 95%, at least 96%, at least 97%, at least 98% or at least 99%) identical to SEQ ID NO: 11 or a variant amino acid sequence of SEQ ID NO: 11 with 1 or 2 amino acid substitutions;
v. a HC CDR2 comprising an amino acid sequence of SEQ ID NO: 12 or an amino acid sequence which is at least 90% (e.g., at least 95%, at least 96%, at least 97%, at least 98% or at least 99%) identical to SEQ ID NO: 12 or a variant amino acid sequence of SEQ ID NO: 12 with 1 or 2 amino acid substitutions;
vi. a HC CDR3 comprising an amino acid sequence of SEQ ID NO: 13 or an amino acid sequence which is at least 90% (e.g., at least 95%, at least 96%, at least 97%, at least 98% or at least 99%) identical to SEQ ID NO: 13 or a variant amino acid sequence of SEQ ID NO: 13 with 1 or 2 amino acid substitutions.

In various instances, the antibody comprises: a LC variable region comprising an amino acid sequence of SEQ ID NO: 14, an amino acid sequence which is at least 90% (e.g., at least 95%, at least 96%, at least 97%, at least 98% or at least 99%) identical to SEQ ID NO: 14, or a variant amino acid sequence of SEQ ID NO: 14 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the antibody comprises: a HC variable region comprising an amino acid sequence of SEQ ID NO: 15, an amino acid sequence which is at least 90% (e.g., at least 95%, at least 96%, at least 97%, at least 98% or at least 99%) identical to SEQ ID NO: 15, or a variant amino acid sequence of SEQ ID NO: 15 with 1 to 10 (e.g., 1 to 9, 1 to 8, 1 to 7, 1 to 6, 1 to 5, 1 to 4, 1 to 3, 1 or 2) amino acid substitutions.

In exemplary instances, the antibody comprises a light chain comprising an amino acid sequence of SEQ ID NO: 16, an amino acid sequence which is at least 90% (e.g., at least 95%, at least 96%, at least 97%, at least 98% or at least 99%) identical to SEQ ID NO: 16, or a variant amino acid sequence of SEQ ID NO: 16 with 1 to 10 (e.g., 1 to 9, 1 to 8, 1 to 7, 1 to 6, 1 to 5, 1 to 4, 1 to 3, 1 or 2) amino acid substitutions.

In various aspects, the antibody comprises a heavy chain comprising an amino acid sequence of SEQ ID NO: 17, an amino acid sequence which is at least 90% (e.g., at least 95%, at least 96%, at least 97%, at least 98% or at least 99%) identical to SEQ ID NO: 17, or a variant amino acid sequence of SEQ ID NO: 17 with 1 to 10 (e.g., 1 to 9, 1 to 8, 1 to 7, 1 to 6, 1 to 5, 1 to 4, 1 to 3, 1 or 2) amino acid substitutions.

Compositions

The presently disclosed methods relate to compositions comprising recombinant glycosylated proteins. In various aspects, the composition comprises only one type of recombinant glycosylated protein. In various instances, the composition comprises recombinant glycosylated proteins wherein each recombinant glycosylated protein of the composition comprises the same or essentially the same amino acid sequence. In various aspects, the composition comprises recombinant glycosylated proteins wherein each recombinant glycosylated protein of the composition comprises an amino acid sequence which is at least 90% identical to the amino acid sequences of all other recombinant glycosylated proteins of the composition. In various aspects, the composition comprises recombinant glycosylated proteins wherein each recombinant glycosylated protein of the composition comprises an amino acid sequence which is at least 95%, at least 96%, at least 97%, at least 98%, or at least 99% identical to the amino acid sequences of all other recombinant glycosylated proteins of the composition. In various aspects, the composition comprises recombinant glycosylated proteins wherein each recombinant glycosylated protein of the composition comprises an amino acid sequence which is the same or essentially the same (e.g., at least 90% or at least 95%, at least 96%, at least 97%, at least 98%, or at least 99% identical to the amino acid sequences of all other recombinant glycosylated proteins of the composition) but the glycoprofiles of the recombinant glycosylated proteins of the composition may differ from each other. In various aspects, the composition comprises recombinant glycosylated proteins in which a least 80%, of the glycosylated proteins have an unpaired glycan content (high mannose and/or afucosylated) below a specified level (e.g., unpaired content of no more than 90%, 80%, 70%, 60%, 50%, 40% or 30%). In various aspects, the composition comprises recombinant glycosylated proteins in which a least 80% of the glycosylated proteins have an unpaired glycan content (high mannose and/or afucosylated) above a specified level (e.g., unpaired content of at least 90%, 80%, 70%, 60%, 50%, 40% or 30%).

In exemplary aspects, the recombinant glycosylated protein is an antibody fragment and accordingly, the composition may be an antibody fragment composition.

In exemplary aspects, the recombinant glycosylated protein is an antibody protein product and accordingly, the composition may be an antibody protein product composition.

In exemplary aspects, the recombinant glycosylated protein is a Glycosylated Fc Fragment and accordingly, the composition may be a Glycosylated Fc Fragment composition.

In exemplary aspects, the recombinant glycosylated protein is a Glycosylated Fc Fragment antibody product and accordingly, the composition may be a Glycosylated Fc Fragment antibody product composition.

In exemplary aspects, the recombinant glycosylated protein is a chimeric antibody and accordingly, the composition may be a chimeric antibody composition.

In exemplary aspects, the recombinant glycosylated protein is a humanized antibody and accordingly, the composition may be a humanized antibody composition.

In exemplary aspects, the recombinant glycosylated protein is an antibody and the composition is an antibody composition. In various aspects, the composition comprises only one type of antibody. In various instances, the composition comprises antibodies wherein each antibody of the antibody composition comprises the same or essentially the same amino acid sequence. In various aspects, the antibody composition comprises antibodies wherein each antibody of the antibody composition comprises an amino acid sequence which is at least 90% identical to the amino acid sequences of all other antibodies of the antibody composition. In various aspects, the antibody composition comprises antibodies wherein each antibody of the antibody composition comprises an amino acid sequence which is at least 95%, at least 96%, at least 97%, at least 98%, or at least 99% identical to the amino acid sequences of all other antibodies of the antibody composition. In various aspects, the antibody composition comprises antibodies wherein each antibody of the antibody composition comprises an amino acid sequence which is the same or essentially the same (e.g., at least 90% or at least 95%, at least 96%, at least 97%, at least 98%, or at least 99% identical to the amino acid sequences of all other antibodies of the antibody composition) but the glycoprofiles of the antibodies of the antibody composition may differ from each other. In exemplary aspects, the antibody composition comprises a heterogeneous mixture of different glycoforms of the antibody. In various instances, the antibody composition may be characterized in terms of its relative unpaired glycan content, e.g., relative unpaired HM glycan content and/or its relative unpaired AF glycan content. Optionally, the antibody composition may be characterized in terms its content of other types of glycans, e.g., galactosylated glycoforms, fucosylated glycoforms, and the like.

In various aspects, each antibody of the antibody composition is infliximab. In various aspects, each antibody of the antibody composition is trastuzumab.

In exemplary aspects, the antibody composition comprises a heterogeneous mixture of different glycoforms of the antibody. In various instances, the antibody composition may be characterized in terms of its relative unpaired HM glycan content and/or its relative unpaired AF glycan content. In various aspects, the antibody composition is described in terms of % unpaired HM glycan and/or % unpaired afucosylated glycan. Optionally, the antibody composition may be characterized in terms its content of other types of glycans, e.g., galactosylated glycoforms, fucosylated glycoforms, and the like.

In exemplary embodiments, the composition is combined with a pharmaceutically acceptable carrier, diluent or excipient. Accordingly, provided herein are pharmaceutical compositions comprising the recombinant glycosylated protein composition (e.g., the antibody composition or antibody protein product composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

In exemplary embodiments, the antibodies of the antibody composition are expressed by glycosylation competent cells in cell culture as described herein.

Additional Production Processes

The methods of producing antibody compositions disclosed herein, in various aspects, comprise additional processes. For example, in some aspects, the methods comprise upstream or downstream processing involved in producing, purifying, and formulating a recombinant glycosylated protein, e.g., an antibody. Optionally, the downstream processing comprises any downstream processing described herein or known in the art. See, e.g., Downstream Processing, herein. In exemplary embodiments, the method comprises generating host cells that express a recombinant glycosylated protein (e.g., antibody). The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells or BHK cells). Such host cells are described in the art. See, e.g., Frenzel, et al., Front Immunol 4: 217 (2013) and herein under “Cells.” For example, the methods comprise, in some instances, introducing into host cells a vector comprising a nucleic acid comprising a nucleotide sequence encoding the recombinant glycosylated protein, or a polypeptide chain thereof.

In exemplary aspects, the methods comprise maintaining cells, e.g., glycosylation-competent cells in a cell culture. Accordingly, the methods may comprise carrying out any one or more steps described herein in Maintaining Cells In A Cell Culture.

In exemplary embodiments, the methods disclosed herein comprise isolating and/or purifying the recombinant glycosylated protein (e.g., recombinant antibody) from the culture. In exemplary aspects, the method comprises chromatography including, but not limited to, e.g., affinity chromatography (e.g., protein A affinity chromatography), ion exchange chromatography, and/or hydrophobic interaction chromatography. In exemplary aspects, the method comprises producing crystalline biomolecules from a solution comprising the recombinant glycosylated proteins.

The methods of the disclosure, in various aspects, comprise preparing a composition, including, in some aspects, a pharmaceutical composition, comprising the purified recombinant glycosylated protein. Such compositions are discussed herein.

Maintaining Cells In A Cell Culture

With regard to the methods of producing a protein or antibody composition of the present disclosure, the antibody composition may be produced by maintaining cells in a cell culture (e.g., maintaining a cell culture). The cell culture may be maintained according to any set of conditions suitable for production of a recombinant glycosylated protein. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 10⁶ cells/mL in 1.5 L medium. As described herein, a clone may be selected to produce an selected relative unpaired and paired glycan content (for example an unpaired glycan content lower or higher than a control). It will be understood that cells derived from the clone may be cultured for the production of protein or antibody compositions as described herein.

In exemplary aspects, the methods of the disclosure comprise maintaining the glycosylation-competent cells in a cell culture medium at a pH of about 6.85 to about 7.05, e.g., in various aspects, about 6.85, about 6.86, about 6.87, about 6.88, about 6.89, about 6.90, about 6.91, about 6.92, about 6.93, about 6.94, about 6.95, about 6.96, about 6.97, about 6.98, about 6.99, about 7.00, about 7.01, about 7.02, about 7.03, about 7.04, or about 7.05.

In exemplary aspects, the methods comprise maintaining the cell culture at a temperature between 30° C. and 40° C. In exemplary embodiments, the temperature is between about 32° C. to about 38° C. or between about 35° C. to about 38° C.

In exemplary aspects, the methods comprise maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, about 225, about 250, about 275, about 300, about 325, about 350, about 375, about 400, about 425, about 450, about 475, or about 500.

In exemplary aspects, the methods comprise maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

The cell culture is maintained in any one or more culture medium. In exemplary aspects, the cell culture is maintained in a medium suitable for cell growth and/or is provided with one or more feeding media according to any suitable feeding schedule. In exemplary aspects, the method comprises maintaining the cell culture in a medium comprising glucose, fucose, lactate, ammonia, glutamine, and/or glutamate. In exemplary aspects, the method comprises maintaining the cell culture in a medium comprising manganese at a concentration less than or about 1 μM during the initial cell culture period. In exemplary aspects, the method comprises maintaining the cell culture in a medium comprising about 0.25 μM to about 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb. In exemplary aspects, the cell culture medium comprises mannose. In exemplary aspects, the cell culture medium does not comprise mannose.

In exemplary embodiments, the type of cell culture is a fed-batch culture or a continuous perfusion culture. However, the methods of the disclosure are advantageously not limited to any particular type of cell culture.

The cells maintained in cell culture may be glycosylation-competent cells. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

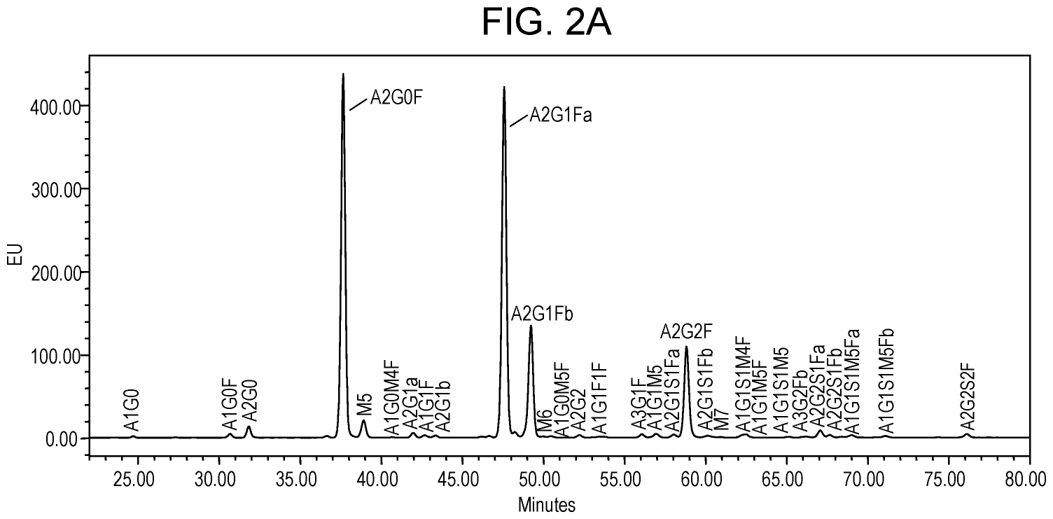

In exemplary embodiments, the glycosylation-competent cells are not genetically modified to alter the activity of an enzyme of the de novo pathway or the salvage pathway. These two pathways of fucose metabolism are shown in FIG. 3. In exemplary embodiments, the glycosylation-competent cells are not genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are not genetically modified to knock-out a gene encoding FX.

In exemplary embodiments, the glycosylation-competent cells are not genetically modified to alter the activity β(1,4)-N-acetylglucosaminyltransferase III (GNTIII) or GDP-6-deoxy-D-lyxo-4-hexulose reductase (RMD). In exemplary aspects, the glycosylation-competent cells are not genetically modified to overexpress GNTIII or RMD.

In exemplary embodiments, the glycosylation-competent cells are genetically modified to alter the activity of an enzyme of the de novo pathway or the salvage pathway.

The following examples are given merely to illustrate the present invention and not in any way to limit its scope.

### EXAMPLES

### Example 1

This example describes an exemplary method of determining an N-linked glycosylation profile (glycan profile) for a monoclonal antibody.

The purpose of this analytical method is to determine the N-linked glycosylation profile of an antibody in samples comprising the antibody by hydrophilic interaction liquid chromatography (HILIC) ultra high performance liquid chromatography (UHPLC) glycan map analysis. This glycan map method is a quantitative analysis of the N-linked glycan distribution of the antibody and comprises three steps: (1) release and label N-linked glycans from reference and test samples using PNGase F and a fluorophore that can specifically derivatize free glycan, (2) load samples within the validated linear range onto a HILIC column, the labeled N-linked glycans are separated using a gradient of decreasing organic solvent, and (3) monitor elution of glycan species with fluorescence detector.

The standard and test samples are prepared by carrying out the following steps: (1) dilute samples and controls with water, (2) add PNGase F and incubate the samples and controls to release N-linked glycans, (3) mix with fluorophore labeling solution using a fluorophore such as 2-aminobenzoic acid. Vortex and incubate the samples and controls, (4) centrifuge down to pellet protein and remove supernatant, and (5) dry and reconstitute labeled glycans in the injection solution.

The solutions used in this assay are a Mobile Phase A (100 mM ammonium formate, target pH 3.0) and a Mobile Phase B (acetonitrile). The equipment used to perform steps of the method has the following capabilities:

Equipment capabilities:

HPLC system
Fluorescence detector set to appropriate
excitation/emission wavelength optimized to labeling
fluorophore
Data collection system
Temperature-controlled autosampler
Hydrophilic interaction column

The instrument settings for HPLC using a hydrophilic interaction analytical BEH Glycan 1.7 μm column (2.1 mm ID×150 mm) and 2-aminobenzoic acid fluorophore labeling method are provided below:

| | |
|---|---|
| Target sample load | 2 μL |
| Column heater set point | 35° C. |
| Auto-sampler set point | 10° C. |
| Detection | Excitation 360 nm |
| | Emission 425 nm |

The mobile phase gradient example is provided below:

| Time | Flow Rate | Mobile Phase A | Mobile Phase B |
|---|---|---|---|
| (minutes) | (mL/minute) | (%) | (%) |
| 0.0 | 0.25 | 22.0 | 78.0 |
| 111.2 | 0.25 | 40.1 | 59.9 |
| 117.9 | 0.20 | 90.0 | 10.0 |
| 124.5 | 0.20 | 90.0 | 10.0 |
| 129.1 | 0.25 | 22.0 | 78.0 |
| 155 | 0.25 | 22.0 | 78.0 |

Reports of the results comprise the following format:

Report area % for high mannose and afucosylation*

% High mannose (% HM) = % M5 + % M6 + % M7
% Afucosylation (% Afuc) = % A1G0 + % A2G0 + % A2G1(a) +
% A2G1(b) + % A2G2 + % A1G1M5

*Calculation formulas depend on presence of individual high mannose and afucosylated species

An example of representative glycan map chromatogram is shown in FIG. 2A (full scale view) and FIG. 2B (expanded scale view).

### Example 2

This example describes the glycan pairing analyses of two antibodies.

The ADCC activity of IgG molecules with an Fc region is influenced by the structure and composition of the oligosaccharides at the consensus glycosylation site. It is well-known that the lack of core fucose on an Fc chain increases ADCC activity by more than an order of magnitude compared to a corresponding Fc chain that comprises the core fucose. This effect of afucosylation on ADCC activity is due to the lack of fucose, which, if present, sterically hinders the binding interaction between the Fc chain to an Fc receptor, e.g., FcγRIIIa, expressed on the surface of effector cells. Accordingly, afucosylated and high mannose glycan groups have been reported as directly influencing ADCC. The existing knowledge surrounding Fc-FcγRIIIa interactions focuses on the binding of a single Fc chain to the receptor. However, protein therapeutic molecules, including IgG and fusion proteins comprising IgG Fc regions, contain two Fc chains, each of which is glycosylated.

In this study, the paired glycan content and unpaired glycan content was determined for two different antibody compositions: (1) a composition comprising Antibody A, a chimeric monoclonal IgG1 antibody comprising human constant and murine variable regions that binds to TNFα and (2) a composition comprising Antibody B, a recombinant IgG1 kappa, humanized monoclonal antibody that binds to human epidermal growth factor receptor protein (HER2). The influence of select glycans on FcγRIIIa interactions and ADCC activity were analyzed. In this example, the paired glycans and unpaired glycans for each antibody were quantified. Example 3 describes the influence of selected glycans on ADCC and Example 4 describes the correlation between ADCC and FcγRIIIa binding activities.

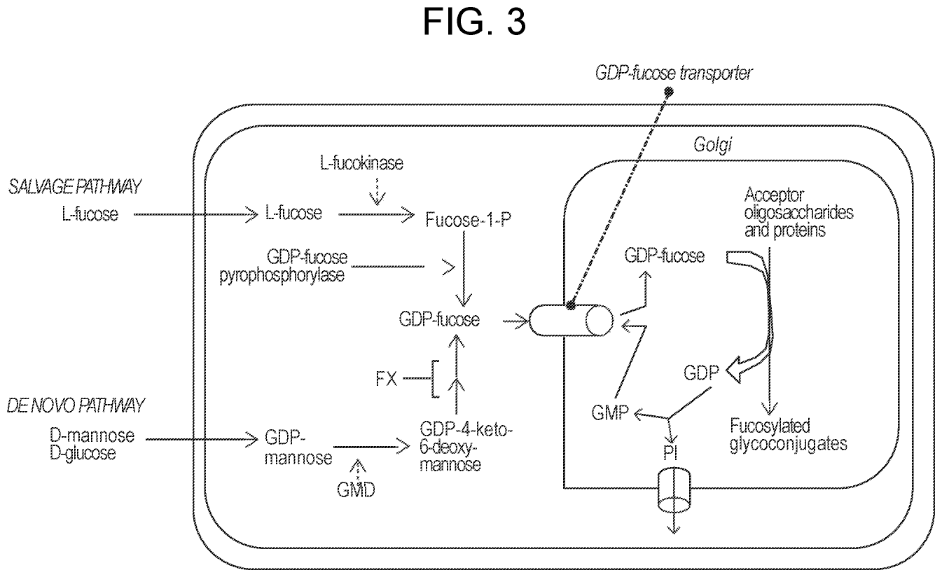

The method used to measure the levels of unpaired or paired glycans comprised three steps: an antibody cleavage step, a chromatographic separation step, and a mass spectrometry-based detection step. Briefly, samples of an antibody composition comprising Antibody A or Antibody B were treated with FabALACTICA® (FL) enzyme (Genovis Inc., Cambridge, MA) which cleaves the heavy chain of IgG1 antibodies above the hinge to create Fab and Fc antibody fragments (FIG. 5). The FL enzyme cleaves between the second and third amino acid of the IgG1 heavy chain sequence KTHTCPP (SEQ ID NO: 1). The resulting Fab fragments and glycosylated Fc fragments are subsequently separated and characterized by HILIC in normal phase mode with water/organic with ion-pairing reagent as solvents and mass spectrometry-based detection. Liquid chromatography (LC) separation was carried out on Waters Acquity UPLC Glycoprotein Amide column over a course of 95 min with mobile phase composition 20:80 of water and acetonitrile with 0.1% TFA. At 1 min, a linear gradient of 0.44 ml/min was applied for the next 74 min for separating glycan pairs based on their interaction with the stationary phase. Mass spectrometry (MS) detection was carried out using an Agilent 6545XT QToF MS with an Agilent Jet Stream (AJS) ion source with settings of: capillary voltage—4500 V; drying gas—11 mL/min; nebulizer pressure—25 psi and gas temperature—340° C. using a mass range of 1000-3000 m/z. Deconvolution was performed using Bioconfirm B.09.00 (Agilent) using S/N of 30 and output mass range set to 40-60 kDa.

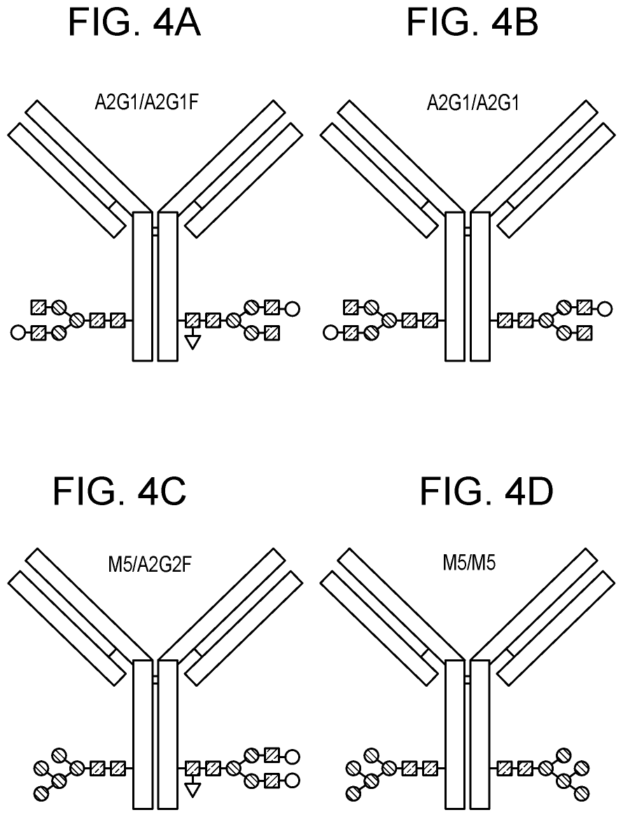

The N-linked glycosylation profile (glycan profile) or glycan map showing paired glycans and unpaired glycans of Antibody A is shown FIG. 6A, and the glycan profile or glycan map showing paired glycans and unpaired glycans of Antibody B is shown FIG. 6B. The abundance of individual glycan pairs was determined as % of relative area under the corresponding peak in the chromatogram as follows:

% Peak Area=Peak Area/Total Peak Area×100

A listing of the glycan pairs in the order of abundance for Antibody A is provided in Table 1 and a listing of the glycan pairs in the order of abundance for Antibody B is provided in Table 2. In each table, species shown in bold-italicized text are structural isomers.

TABLE 1

| Glycan pairs | Antibody A |
|---|---|
| A2G0F/A2G0F | 36.17 |
| A2G0F/A2G1F | 13.67 |
| A2G0F/A2G1F | 10.08 |
| A2G0/A2G1F | 5.58 |
| A2G1F/A2G1F | 4.33 |
| A2G1F/A2G1F | 4.25 |
| A2G0/A2G0F | 3.42 |
| A1G0F/A2G0F | 3.35 |
| A1G0F/A2G1F | 1.94 |
| A2G1F/A2G1F | 1.88 |
| A2G1F/A2G2F | 1.31 |
| A2G1/A2G1 | 0.91 |
| M5/A2SG1F | 0.91 |
| A2G1/A2G1F or A2G0/A2G2F | 1.12 |
| M5/M5 | 0.73 |
| M5/A2G0F | 0.69 |
| M5/A2SG2F or M6/A2SG1F | 0.67 |
| A1G0/A2G0F or A2G0/A1G0F | 0.55 |
| A2G1F/A2G2F | 0.55 |
| M7/A2G1F | 0.54 |
| A2G1/A2G2 | 0.43 |
| A2G1/A2G2F | 0.39 |
| A2G0/A2G0 | 0.30 |
| A2G2/A2G2F | 0.28 |
| A2G2F/A2G2F | 0.28 |
| M3/A2G0F or M3F/A2G0 | 0.21 |
| A1G1F/A1G1M4F | 0.20 |
| A1G0F/A2G2F | 0.18 |
| M3F/A2G0F | 0.18 |
| M6/A2SG2F or M7/A2SG1F or A1SG1M5/A1G1M4 | 0.29 |
| A2G0F/A2SG2F | 0.14 |
| A2G0F/M7 or A2G1F/M6 | 0.12 |
| A1G1F/A1G0M5 | 0.11 |
| A1G1F/A2G2F | 0.11 |
| M3/A1G0M5 or A1G0/M5 | 0.10 |
| A2G1F/AS2G2F | 0.10 |
| M3/A2G0 or A1G0/A1G0 | 0.07 |
| A2G1F/A2SG2F | 0.05 |
| A2SG1F/A2SG2F or A2G1F/A2SG2F2 | 0.04 |
| M3/M5 | 0.03 |
| A2G2F/A2SG2F | 0.03 |
| M3F/A1G0 or M3/A1G0F | 0.03 |
| A2G0F/A2SG2F2 | 0.02 |
| A1G1F/A1G1F or A1G0F/A1G1M4F | 0.02 |

TABLE 2

| Glycan pairs | Antibody B |
|---|---|
| A2G0F/A2G1F | 22.89 |
| A2G0F/A2G0F | 17.41 |
| A2G1F/A2G1F | 11.35 |
| A2G1F/A2G1F | 10.52 |
| A2G0F/A2G1F | 9.99 |
| A2G0/A2G1F | 6.91 |
| A2G0/A2G0F | 2.77 |
| A2G1/A2G1F | 2.55 |
| A1G1F/A2G0F or A1G0F/A2G1F | 1.83 |
| A2G1F/A2G2F | 1.80 |
| A1G0F/A2G0F | 1.69 |
| A2G2/A2G1F | 1.14 |
| A1G1/A2G2F or A2G0F/A1G1M5 or A1G1F/A2G2 | 1.02 |
| A2G2F/A2G2F | 0.60 |
| A1G0/A2SG2F or A1G1/A2SG1F | 0.50 |
| M5/M5 | 0.45 |
| A1G1/A2G1F | 0.44 |
| A1G0F/A2G0 or A1G0/A2G0F | 0.42 |
| A1G0F/A2G1 | 0.28 |
| A2G1F/A2SG2F | 0.23 |
| A2G0/A2G0 | 0.23 |
| A2G1F/A2SG1F | 0.23 |
| A1G0F/A2G2F | 0.19 |
| M5/A2G0F | 0.18 |
| A3G2F/A2G0F | 0.13 |
| A1G0F/A1G0F | 0.08 |
| A2G2F/A2SG2F | 0.07 |
| A2SG1F/A2SG2F | 0.05 |
| A3G2F/A2G1F | 0.04 |
| A1G0/A2G0 | 0.02 |
| A2G0F/A1G1M5 | 0.02 |
| M5/A1G0 | 0.01 |

The relative abundances of high mannose-containing glycan pairs for Antibody A and Antibody B are listed in Table 3, while the relative abundances of afucosylated glycan pairs for Antibody A and Antibody B are listed in Table 4. An “unpaired” status was assigned to those pairs wherein only one Fc chain had a core fucose (F), and a “paired” status was assigned to those pairs wherein neither Fc chain had a core fucose. In general, high mannose-containing pairs comprised one or two high mannose groups, except for the glycan pair involving M3 and A2G0, wherein core fucose was present on one of these glycans (either M3F or A2G0F). Afucosylated glycan pairs comprised an afucosylated glycan on at least one Fc chain. In some instances, the afucosylated glycan pair comprised one high mannose group.

TABLE 3
High Mannose-Containing Glycan Pairs

| | | Antibody A | Antibody B |
|---|---|---|---|
| | Pairing | (% relative | (% relative |
| Glycan pairs | category | abundance) | abundance) |
| M5/M5 | Paired | 0.73 | 0.45 |
| M3/M5* | | 0.03 | |
| # M3/A1G0M5 or A1G0/M5* | | 0.10 | |
| M5/A2SG1F | Unpaired | 0.91 | |
| M5/A2G0F | | 0.69 | 0.18 |
| M5/A2SG2F or M6/A2SG1F | | 0.67 | |
| M7/A2G1F | | 0.54 | |
| # M6/A2SG2F or M7/A2SG1F or | | 0.29 | |
| A1SG1M5/A1G1M4 | | | |
| A2G0F/M7 or A2G1F/M6 | | 0.12 | |

| | | |
|---|---|---|
| Total (sum of % relative abundance for all high mannose | 3.69-4.08 | 0.63 |
| containing glycan pairs) | | |
| % Unpaired (relative to all high mannose containing glycan | 79 | 29 |
| pairs) | | |
| % Unpaired (relative to all high mannose containing glycan | 67 | 17 |
| species) | | |

*Based on assumption that both afucosylated and high mannose species are highly potent, pairs containing a combination of high mannose and afucosylated glycans were included into the Paired group. Mixed A1G0/M5 species were included in both high mannose and afucosylated glycan pairs tables for completeness of assessment of both groups ″low″ and ″high″ pared/unpaired composition range.
# Note:
Due to uncertainty in species assignments for glycan pairs in peaks labeled by # low- and high-end estimates were performed and results were presented as range where applicable. For low-end estimate area of peaks labeled by # in Glycan pairs columns were not included to account for possibility that high mannose containing species were not present due to inability to differentiate from other species potentially present in the same peak.
Note:
Hybrid species and M3 were not included into ″High Mannose″ group, but counted under ″Afucosylated″ group

TABLE 4
Afucosylated Glycan Pairs

| | | Antibody A | Antibody B (% relative |
|---|---|---|---|
| Glycan pairs peaks | Pairing category | (% relative abundance) | abundance) |
| A2G0/A2G0 | Paired | 0.30 | 0.23 |
| A2G1/A2G1 | | 0.91 | |
| # M3/A2G0 or A1G0/A1G0* | | 0.07 | |
| A2G1/A2G2 | | 0.43 | |
| A1G0/A2G0 | | | 0.02 |
| M3/A1G0M5 or A1G0/M5 | | 0.10 | 0.01 |
| M3F/A1G0 or M3/A1G0F | Unpaired | 0.03 | |
| M3/A2G0F or M3F/A2G0 | | 0.21 | |
| A1G0/A2G0F or A2G0/A1G0F | | 0.55 | 0.42 |
| A2G0/A2G0F | | 3.42 | 2.77 |
| A2G0/A2G1F | | 5.58 | 6.91 |
| A2G1/A2G1F | | 1.12 | 2.55 |
| A2G1/A2G2F | | 0.39 | 1.14 |
| A2G2/A2G2F | | 0.28 | |
| A1G1/A2G1F | | | 0.44 |
| A1G1/A2G2F or A2G0F/A1G1M5 or | | | 1.02 |
| A1G1F/A2G2 | | | |
| A1G0/A2G2SF or A1G1/A2G1SF | | | 0.50 |
| A1G0F/A2G1 | | | 0.28 |
| A2G0F/A1G1M5 | | | 0.02 |
| # M6/A2SG2F or M7/A2SG1F or | | 0.29 | |
| A1SG1M5/A1G1M4* | | | |

| | | |
|---|---|---|
| Total (sum of % relative abundance for all afucosylation | 12.10-13.68 | 16.31 |
| containing glycan pairs) | | |
| % Unpaired (relative to all afucosylation containing | 85-88 | 98 |
| glycan pairs) | | |
| % Unpaired (relative to all afucosylation containing | 77 | 97 |
| glycan species) | | |

*Based on assumption that both afucosylated and high mannose species are highly potent, pairs containing a combination of high mannose and afucosylated glycans were included into the Paired group. M3 glycan was counted under afucosylated group. Mixed A1G0/M5 species were included in both high mannose and afucosylated glycan pairs tables for completeness of assessment of both groups ″low″ and ″high″ pared/unpaired composition range.
# Note:
Due to uncertainty in species assignments for glycan pairs in peaks labeled by # low- and high-end estimates were performed and results were presented as range where applicable. For low-end estimate area of peaks labeled by # in Glycan pairs columns were not included to account for possibility that afucosylation containing species were not present due to inability to differentiate with other species.

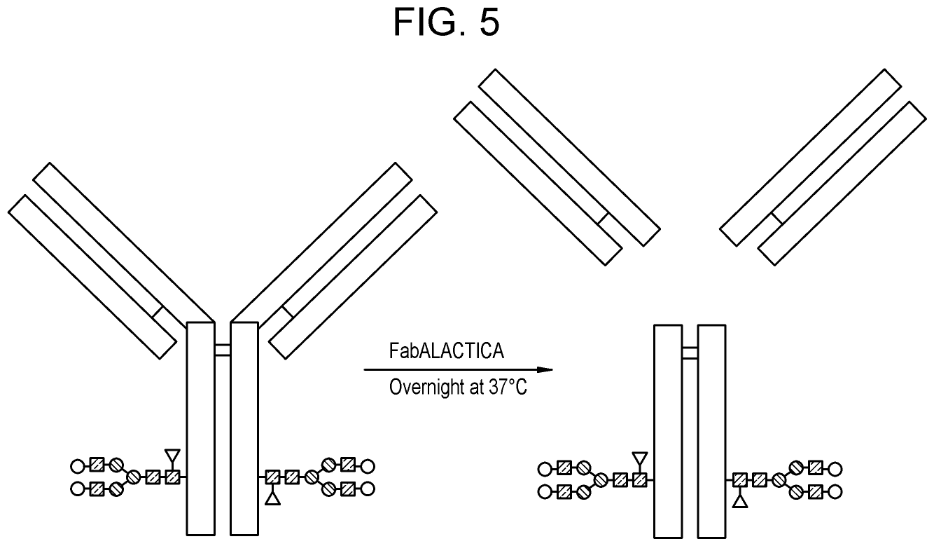

A graph of the relative abundance of unpaired afucosylated glycans (%) and relative abundance of unpaired high mannose glycans (%) for Antibody A and Antibody B is shown in FIG. 7. The relative abundance of unpaired afucosylated glycans (%) was calculated by dividing the percentage of unpaired afucosylated glycans by the sum of the percentage of unpaired afucosylated glycans and the percentage of paired afucosylated glycans and multiplying by 100%. The relative abundance of unpaired high mannose glycans was calculated by dividing the percentage of unpaired high mannose glycans by the sum of the percentage of unpaired high mannose glycans and the percentage of paired high mannose glycans and multiplying by 100%. As shown in this figure, both antibodies (Antibody A and Antibody B) comprised a high amount of relative unpaired afucosylated glycans. While Antibody A had a high amount of relative unpaired high mannose glycans, relative unpaired high mannose glycans represented a smaller percentage for Antibody B.

### Example 3

This example describes the ADCC analyses of two antibodies.

ADCC activity levels (expressed as a % relative value) for samples comprising Antibody A or Antibody B were determined using a quantitative cell-based assay that measures the ability of the antibody to mediate cell cytotoxicity in a dose-dependent manner of target cells stably expressing target antigens for Antibody A or Antibody B while engaging FcγRIIIA (158V) receptors on NK92-M1 effector cells via the antibody Fc domain. These events lead to the activation of the effector cells and destruction of the target cells via exocytosis of the cytolytic granule complex perforin/granzyme. A schematic of the ADCC assay for Antibody A is provided in FIG. 8A and a representative dose-response curve for the ADCC assay is shown in FIG. 8B. In FIG. 8B, each dose point is a mean±standard deviation of 3 replicates and the assay signal=fluorescence.

Two series of antibody samples (one series for Antibody A and another for Antibody B) having increasing antibody concentrations were made. The abundance of high mannose glycans and afucosylated glycans for each sample of each series was measured and recorded. ADCC activity levels (expressed as a %) for each sample of each series were determined through the above quantitative cell-based assay. Briefly, target cells were labeled with calcein-acetoxymethyl (calcein-AM), which readily enters the cells and is subsequently cleaved by intercellular esterases and trapped within the cells. When target cells are lysed, fluorescent calcein is released into the medium. The level of calcein released from lysed target cells was determined by measuring the fluorescence of the reaction supernatant in an Envision (Perkin Elmer) fluorescence plate reader. Each assay was performed in triplicate with the mean and standard deviation reported. The data were fitted to the mean fluorescence values using a constrained 4 parameter fit using SoftMaxPro software and reported as percentage ADCC activity relative to a reference standard as calculated by the EC50 standard/EC50 sample ratio.

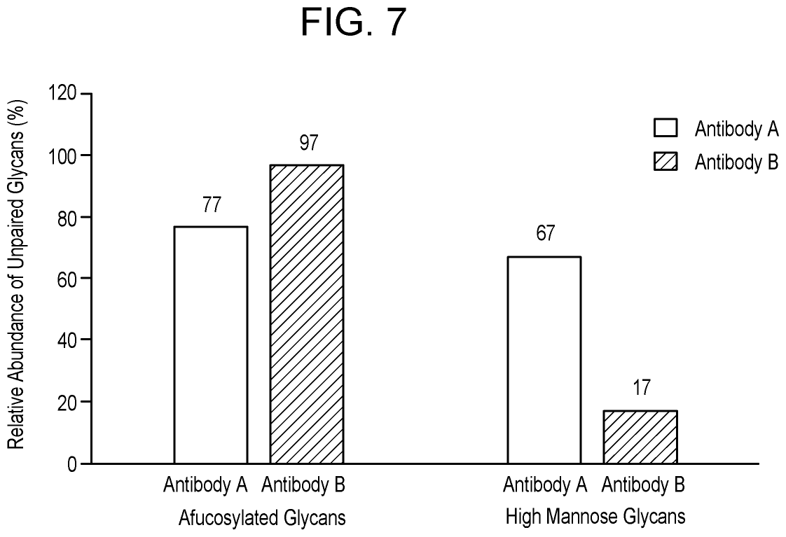

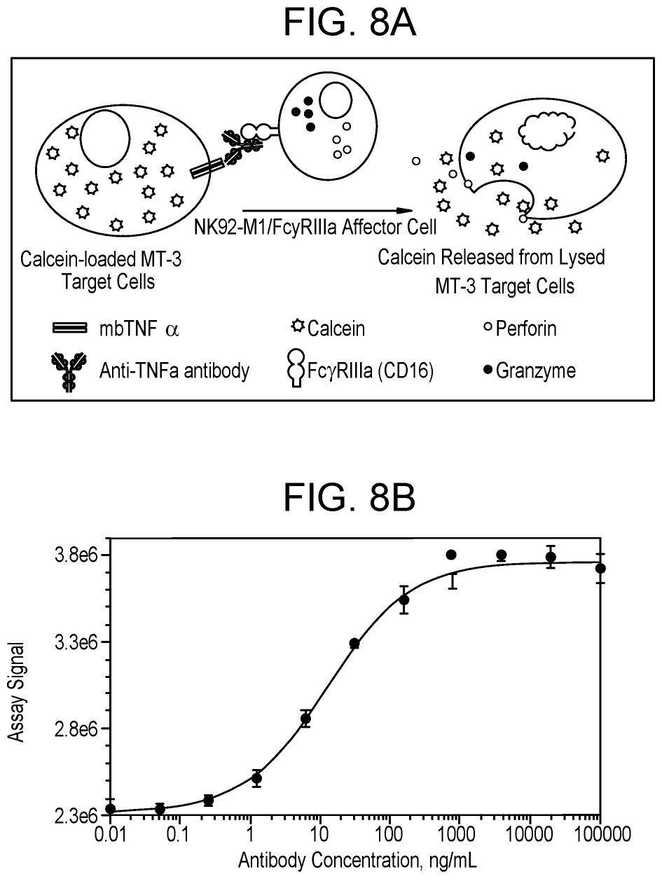

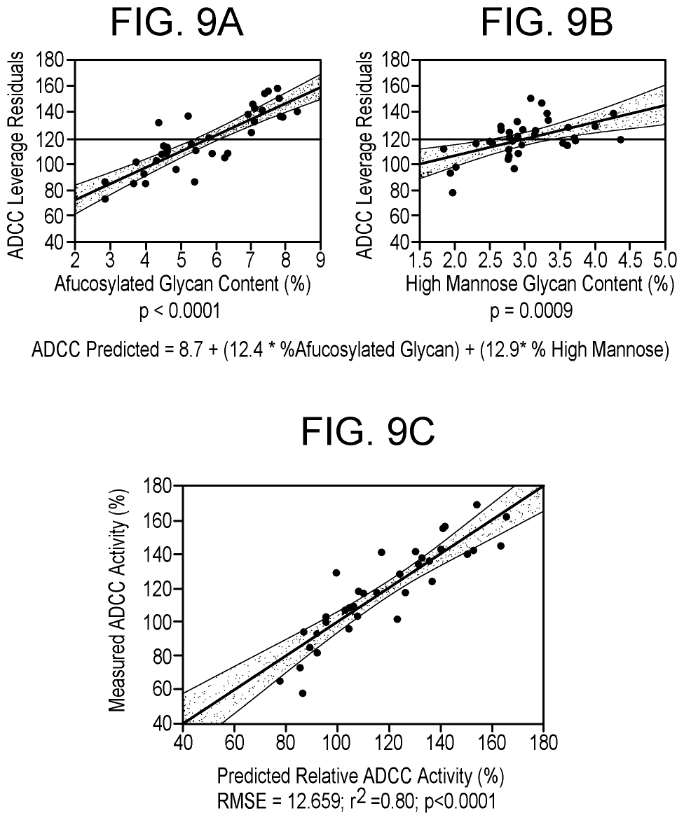

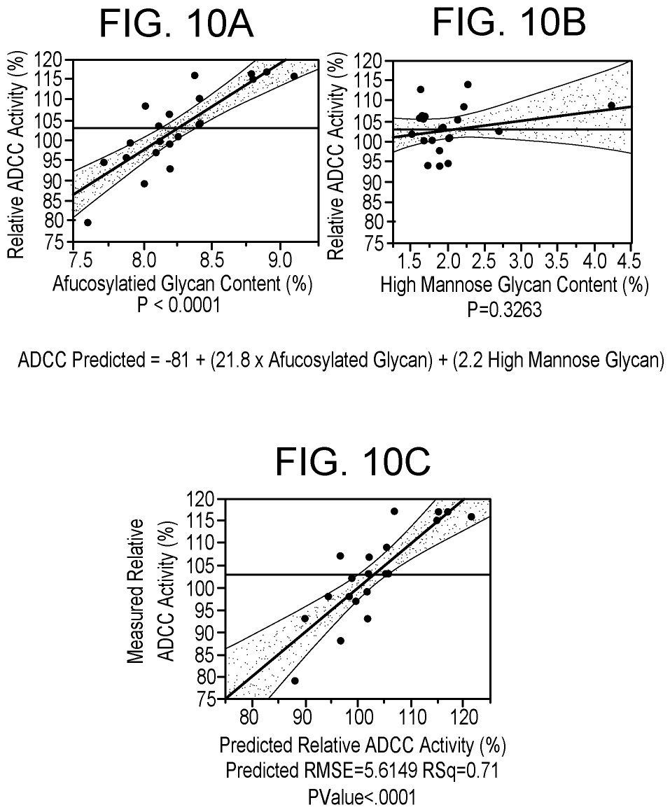

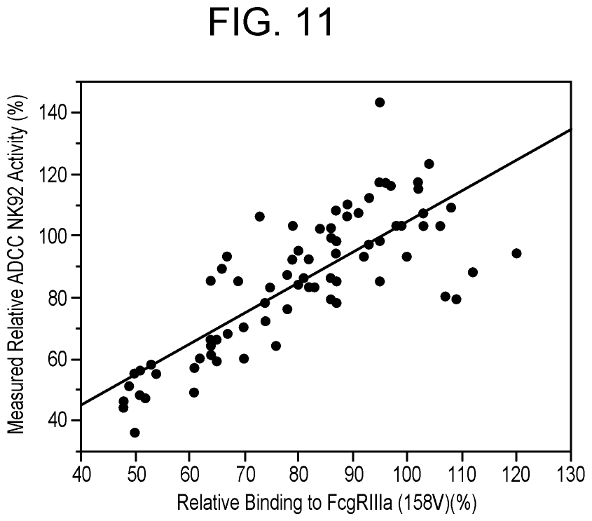

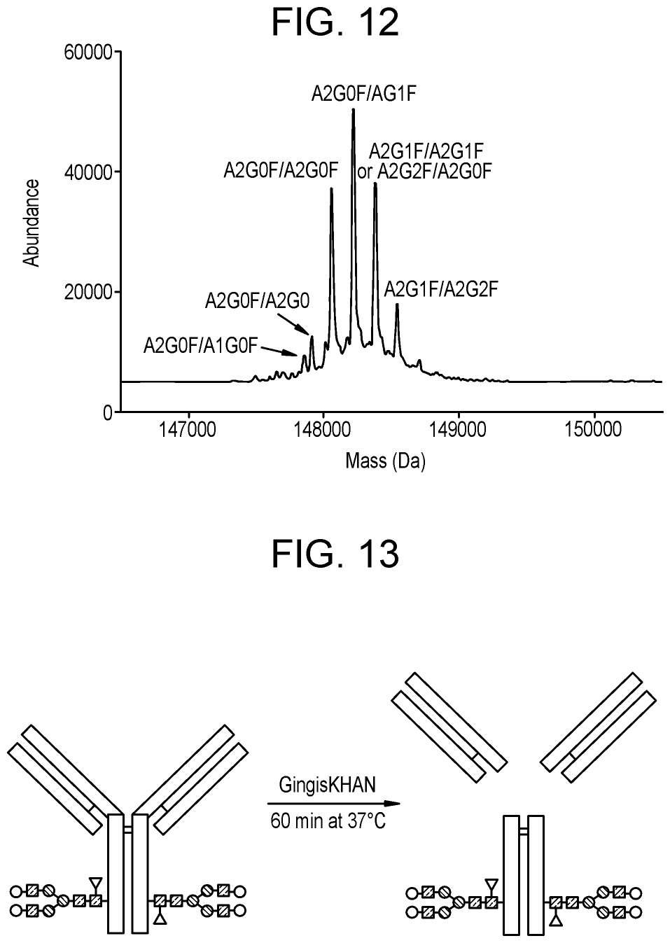

The data for measured ADCC activity level, high mannose glycan content, and afucosylated glycan content were analyzed using JMP suite of computer programs for statistical analysis (SAS Institute, Cary, NC). The results of the assays are shown in FIGS. 9A-9C for Antibody A and FIGS. 10A-10C for Antibody B.

A glycan-ADCC statistical model for Antibody A is provided in FIGS. 9A and 9B wherein FIG. 9A is an ADCC leverage plot for afucosylated glycans and FIG. 9B is a leverage plot for high mannose glycans. The best fit line is the solid diagonal line in the middle of the shaded area. The relationship between % ADCC and the measured glycans for Antibody A may be described by Equation 1:

Predicted % ADCC=8.7+(12.4*% Afucosylated Glycans)+(12.9*% High Mannose Glycans) [Equation 1]

Plugging the measured values for % high mannose and % afucosylated glycans into Equation 1, a predicted % ADCC value was calculated for each sample. The actual % ADCC (as measured in the cell-based assay) was plotted against the predicted % ADCC (as calculated by Equation 1) and the plot is provided as FIG. 9C. Statistical parameters, including Root Mean Square Error (RMSE), r², and p-value, are shown in FIG. 9C. These results suggested that Equation 1 predicted the actual (measured) ADCC with accuracy and underlines the statistically significant direct correlation between afucosylated glycans, high mannose, and ADCC (p<0.0001). Higher levels of afucosylated glycans and high mannose result in higher ADCC activity. The leverage of afucosylated glycans and the leverage of high mannose on ADCC activity were highly similar (12.4 and 12.9, respectively).

The same analyses were applied to the data for Antibody B samples. A glycan-ADCC statistical model of the data for Antibody B is provided in FIGS. 10A and 10B wherein FIG. 10A is an ADCC leverage plot for afucosylated glycans and FIG. 10B is an ADCC leverage plot for high mannose glycans. The best fit line is the solid diagonal line in the middle of the shaded area. As shown in FIG. 10A, the association between afucosylated glycan content and actual ADCC level was statistically significant (p<0.0001), whereas the association between high mannose glycan content and actual ADCC level was not significant (p>0.01). Therefore, the relationship between % ADCC and the measured glycans for Antibody B was dependent on the afucosylated glycan content but not high mannose content. The relationship between % ADCC and the measured afucosylated glycans for Antibody B may be described by Equation 2:

Predicted % ADCC=−81+(21.8*% Afucosylated Glycans)+(2.2 High Mannose Glycans) [Equation 2]

Plugging the measured value for % afucosylated glycans into Equation 2, a predicted % ADCC value was calculated for each sample. The actual % ADCC (as measured in the cell-based assay) was plotted against the predicted % ADCC (as calculated by Equation 2) and the plot is provided as FIG. 10C. Statistical parameters, including Root Mean Square Error (RMSE), r², and p-value, are shown in FIG. 10C. These results suggested that Equation 2 predicted the actual (measured) ADCC with accuracy and underlines the statistically significant correlation between afucosylated glycans and ADCC (p<0.0001). Higher levels of afucosylated glycans result in higher ADCC activity. The effect of high mannose on ADCC was weak and not statistically significant.

The leverage of afucosylated glycans and high mannose on ADCC level as detailed in Equations 1 and 2 was compared to the relative % of unpaired afucosylated glycans and relative % of unpaired high mannose glycans for each antibody as detailed in FIG. 7 (Table 5).

TABLE 5

| | | | ADCC |
|---|---|---|---|
| Glycan group | Antibody | % Unpaired* | leverage** |
| Afucosylated | Antibody A | 77 | 12.4 |
| | Antibody B | 97 | 21.8 |
| High Mannose | Antibody A | 67 | 12.9 |
| | Antibody B | 17 | 2.2*** |

*% unpaired data taken from FIG. 7.
**ADCC leverage corresponds to the value multiplied by the % indicated glycan in Equations 1 and 2 and corresponds to the percent change in relative ADCC in response to 1% change in glycan group abundance.
***Antibody B high mannose leverage coefficient did not reach statistical significance

Interestingly, as supported by Table 5, the leverage of glycan content on ADCC is consistent with the relative abundance of the unpaired glycan type. For Antibody A, the relative abundance of both unpaired glycan types (unpaired afucosylated glycans and unpaired high mannose glycans) was relatively high and both glycan types had a significant impact on ADCC. For Antibody B, the relative abundance of only unpaired afucosylated glycans was relatively high and only this type of glycan (afucosylated glycans) significantly impacted ADCC levels. These results suggest that a higher relative percentage of a particular unpaired glycan group correlates with greater impact on ADCC.

These results suggest that target ADCC levels of an antibody may be reflected by the relative unpaired afucosylated glycan content and/or the relative unpaired high mannose glycan content of the antibody, and that target ADCC levels of an antibody may be modified by altering conditions to modify the relative unpaired afucosylated glycan content and/or the relative unpaired high mannose content. This modulation of unpaired glycan composition can be achieved via selection of cell lines and/or selection/adjustment of process conditions to achieve the desired unpaired afucosylated and/or unpaired high mannose glycan content.

### Example 4

This example describes an exemplary method of measuring and the correlation between ADCC activity and FcγRIIIa binding activity.

FcγRIIIa binding activity of Antibody B was quantified using AlphaLISA® assays (Perkin Elmer, Shelton, CT, USA). Recombinant, purified FcγRIIIa glutathione S-transferase (GST)-fusion proteins were generated at Amgen Inc. (Thousand Oaks, CA). The AlphaLISA® assay is an Amplified Luminescent Proximity Homogenous Assay (Alpha) designed to measure the level of FcγRIIIa binding to the Fc portion of IgG1 mAbs. The assay contained two bead types: an acceptor bead and a donor bead. The acceptor beads were coated with glutathione for binding to recombinant human FcγRIIIa-GST. The donor beads were coated with streptavidin for binding to biotinylated human IgG1 (Amgen Inc. Thousand Oaks, CA). In the absence of Antibody B, FcγRIIIa binds to human IgG1. When Antibody B is present at sufficient concentrations to inhibit the binding of FcγRIIIa to the biotinylated human IgG1, a dose-dependent decrease in emission is measured using a plate reader. The sample binding relative to the reference standard was determined using a 4-parameter logistic model fit (SoftMax® Pro Software, Molecular Devices, Sunnyvale, CA, USA). Each sample was tested in 3 independent assays, and the final valid result for a given sample was reported as the mean of the 3 measurements. Results were reported as percent relative binding values.

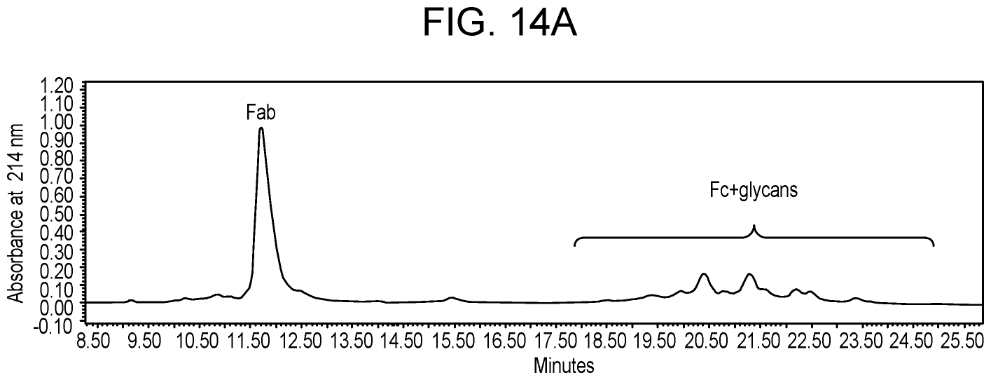

The measured ADCC activity levels obtained in Example 3 for Antibody B were plotted against the measured FcγRIIIa binding activity for Antibody B and the graph is shown in FIG. 11. As shown in this figure, there was good correlation between ADCC activity and FcγRIIIa binding activity for Antibody B.

The measured ADCC activity levels obtained in Example 3 for Antibody A also were plotted against the measured FcγRIIIa binding activity for Antibody A. There was insufficient statistical evidence to conclude whether there was a correlation between ADCC activity and FcγRIIIa binding activity for Antibody A, however. It was contemplated that this lack of a statistical finding may have been attributed to lower purity or greater variability in Fab-mediated target binding activity of samples of Antibody A in the assays that were run. In any case, it will be appreciated that FcγRIIIa binding activity is generally understood to be a surrogate for ADCC representative of Fc-mediated part of the activity, and furthermore, FcγRIIIa binding activity itself is biologically significant.

These results support that the functional activity mediated by Fc and therefore by glycans can be measured by FcγRIIIa binding assay.

### Example 5

This example demonstrates a study of IgG1 Fc glycosylation pairing by intact and middle-down mass spectrometry and a correlation of glycosylation pairing with ADCC activity.

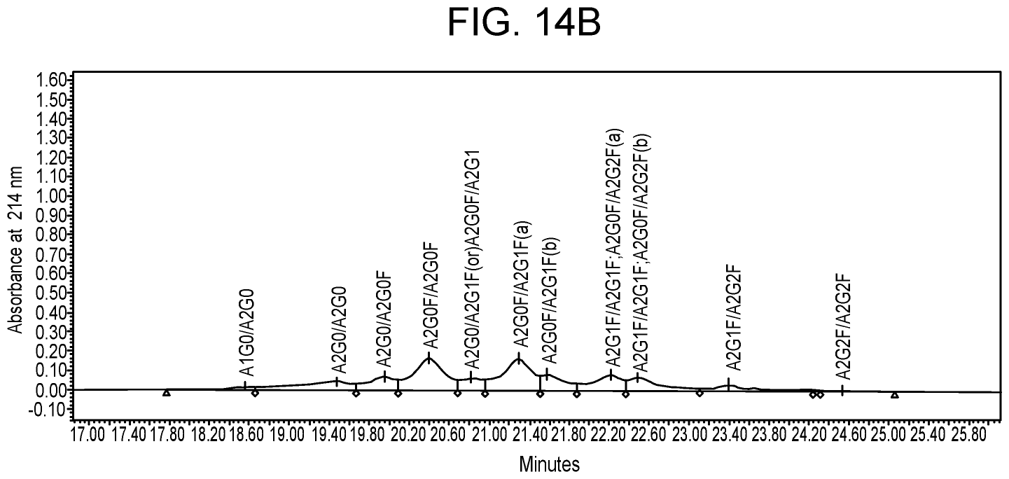

Antibody B was subjected to intact mass analysis by SEC/MS. Briefly, samples were denatured in presence of guanidine hydrochloride and then injected onto the column. Using a mobile phase system composed on water and acetonitrile with 0.1% trifluoacetic acid the sample was eluted. Eluting peaks were then subjected to mass spectrometry detection using an Agilent qTOF instrument. Resulting mass spectra were deconvoluted using Mass Hunter® software (Agilent) and the results are shown in FIG. 12.

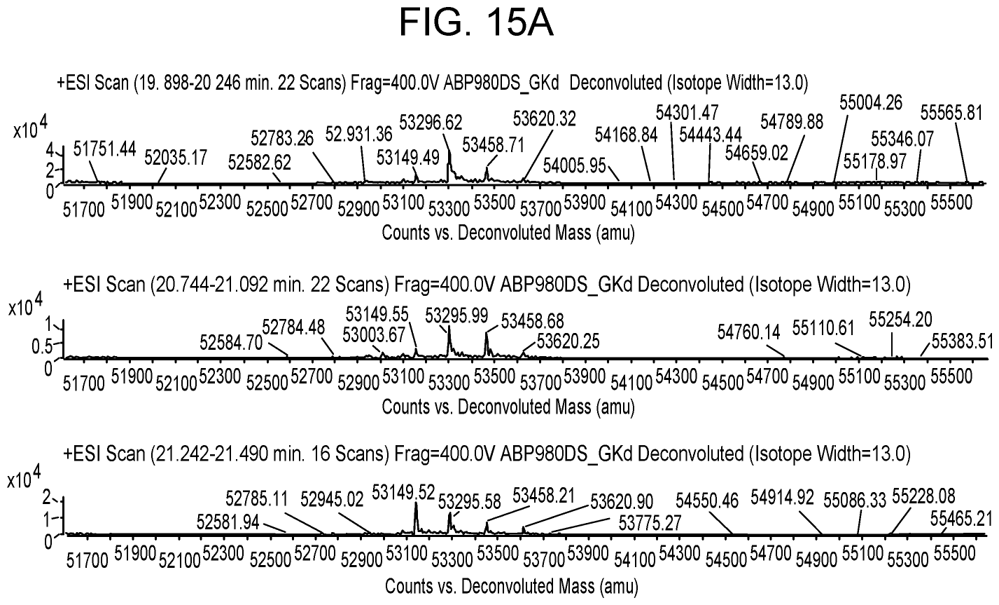

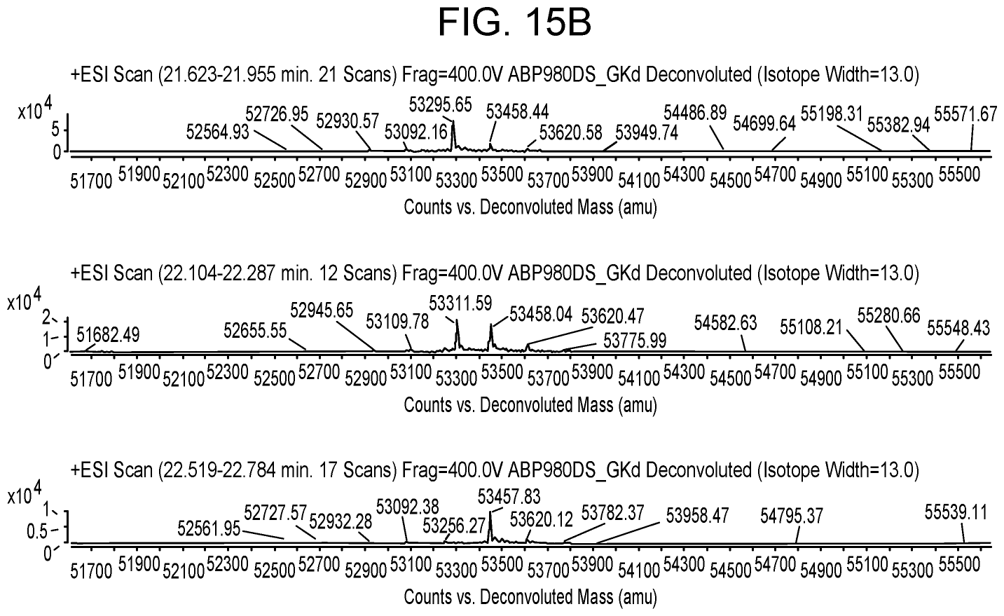

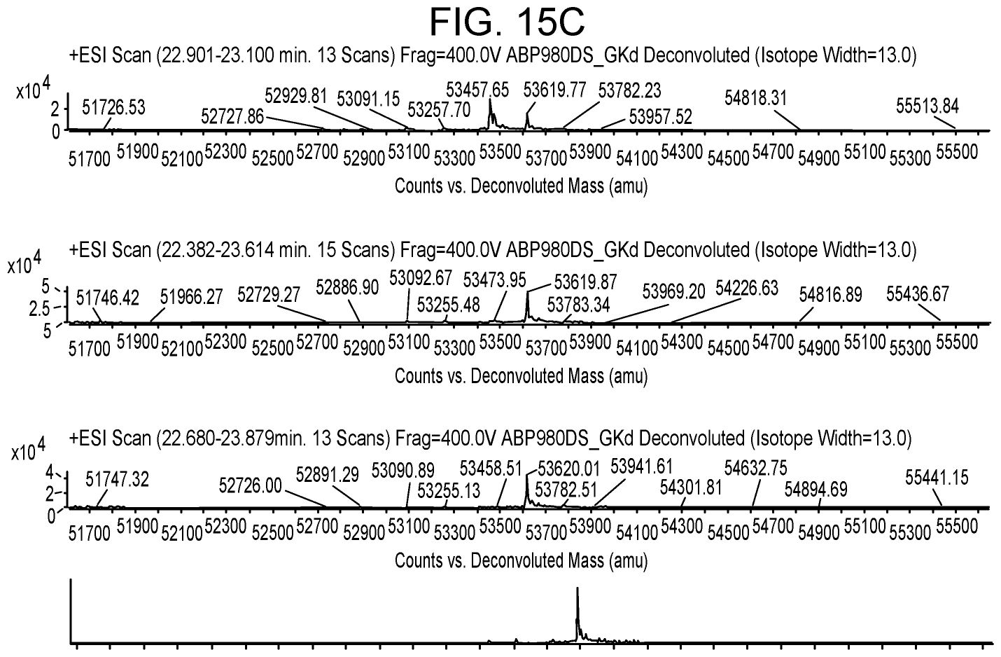

GingisKHAN analysis was carried out by incubating GingisKHAN® Enzyme (Genovis, Lund, Sweden) with Antibody B for 60 min at 37° C. The enzyme is a cysteine protease that digests human IgG1 at a specific site above the hinge to create Fab and Fc antibody fragments (FIG. 13). Chromatographic separation with MS detection was carried out on the digested material to separate the Fab fragments and glycosylated Fc fragments. Exemplary results are shown in FIG. 14A and an expanded view of the peaks of the Fc fragments with labeled glycan pairs are shown in FIG. 14B. FIG. 15 is an example of extracted ion chromatograms showing elution of individual Fc glycan pairs species.

From these studies, it was concluded that intact mass analysis was able to resolve only the glycan pairs with the highest abundance. GingisKHAN analysis was able to provide glycan pairing information.

### Example 6

This example demonstrates FcγRIIIa binding activity correlates with the relative abundance of unpaired afucosylated glycans and the relative abundance of unpaired high mannose glycans.

Antibody C is a monoclonal IgG, antibody that binds to HER2. Though Antibody B and Antibody C bind to the same target (HER2), the antibodies bind to different epitopes. Samples containing Antibody C produced by different clones of two cell lines (five clones total) were used in this study. In a first part of the study, the total abundance of high mannose glycans and afucosylated glycans for each sample was measured as essentially described in Example 1. FcγRIIIa binding activity (expressed as a %) for each sample comprising Antibody C was determined using the assay described in Example 4. The relative abundance of unpaired high mannose glycans and the relative abundance of unpaired afucosylated glycans for each sample was measured and recorded as essentially described in Example 2.

The results are provided in Table 6. Samples of each clone were prepared at least in duplicate (e.g., Samples 1A and 1B are both from Clone 1, etc.), and four samples were run from Clone 2 (Samples 2A, 2B, 2C, and 2D).

TABLE 6

| | | | Relative | Relative | |
|---|---|---|---|---|---|
| | | | Abundance of | Abundance of | |
| | | | Unpaired | Unpaired | |
| | Afucosylated | High Mannose | Afucosylated | High Mannose | FcgRIIIa |
| Sample | Glycans | Glycans | Glycans | Glycans | Binding Activity |
| 1A | 3.1 | 1.6 | 3.0 | 1.3 | 107 |
| 1B | 3.2 | 1.6 | 3.0 | 1.4 | 104 |
| 2A | 3.6 | 1.5 | 3.4 | 1.3 | 111 |
| 2B | 3.2 | 1.5 | 3.1 | 1.4 | 115 |
| 2C | 3.5 | 1.5 | 3.4 | 1.4 | 126 |
| 2D | 3.5 | 1.8 | 3.4 | 1.6 | 129 |
| 3A | 3.9 | 1.9 | 3.7 | 1.9 | 132 |
| 3B | 3.8 | 1.9 | 3.7 | 1.9 | 175 |
| 4A | 5.7 | 1.8 | 5.5 | 0.5 | 156 |
| 4B | 5.9 | 2.1 | 5.7 | 0.5 | 140 |
| 5A | 5.2 | 1.4 | 4.8 | 0.4 | 130 |
| 5B | 5.4 | 1.5 | 5.1 | 0.5 | 132 |

Measured values are reported as %.
Samples 1-8 (1A-3B) were produced by clones of Cell Line 1.
Samples 9-12 (4A-5B) were produced by clones Cell Line 2.

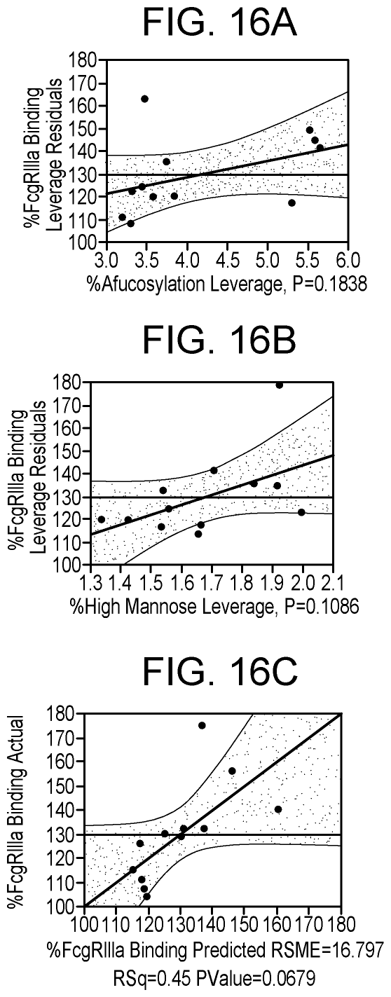

A first glycan-FcγRIIIa binding statistical model for Antibody C is provided in FIGS. 16A and 16B wherein FIG. 16A is an FcγRIIIa binding leverage plot for afucosylated glycans and FIG. 16B is a FcγRIIIa binding leverage plot for high mannose glycans. The best fit line is the solid diagonal line in the middle of the shaded area. The statistical significance of each leverage plot is shown in FIGS. 16A and 16B. As each p-value was greater than or about 0.1, the correlation between total abundance of afucosylated glycans and total abundance of high mannose glycans to FcγRIIIa binding was deemed weak. A predictive equation for a relationship between % FcγRIIIa binding and the measured afucosylated and high mannose glycans for Antibody C could not be derived due to a lack in statistical significance in correlations as shown in the plot provided as FIG. 16C. Statistical parameters, including Root Mean Square Error (RMSE), r², and p-value, are shown in FIG. 16C.

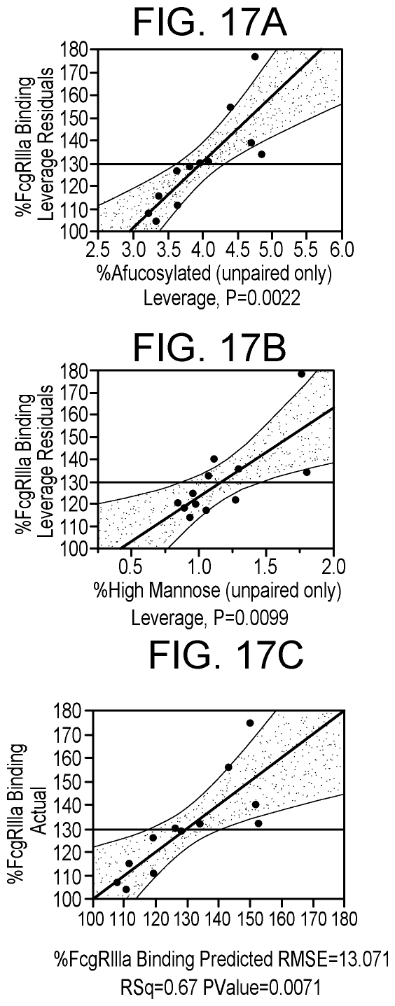

A second glycan-FcγRIIIa binding statistical model for Antibody C is provided in FIGS. 17A and 17B wherein FIG. 17A is an FcγRIIIa binding leverage plot for the relative abundance of unpaired afucosylated glycans and FIG. 17B is a FcγRIIIa binding leverage plot for the relative abundance of unpaired high mannose glycans. The best fit line is the solid diagonal line in the middle of the shaded area and the statistical significance of each leverage plot is shown in FIGS. 17A and 17B. As each p-value was less than 0.05, the correlation between the relative abundance of unpaired afucosylated glycans and relative abundance of unpaired high mannose glycans to FcγRIIIa binding was deemed statistically significant. The relationship between % FcγRIIIa binding and the relative abundance of unpaired afucosylated and relative abundance of unpaired high mannose glycans for Antibody C may be described by Equation 3:

Predicted % FcγRIIIa binding=−32.3+(28.9*% Unpaired Afucosylated Glycans)+(40.2*% Unpaired High Mannose Glycans) [Equation 3]

Applying the measured values for % unpaired afucosylated and % unpaired high mannose glycans into Equation 3, a predicted % FcγRIIIa binding value was calculated for each sample. The actual % FcγRIIIa binding (as measured in the assay) was plotted against the predicted % FcγRIIIa binding (as calculated by Equation 3) and the plot is provided as FIG. 17C. Statistical parameters, including Root Mean Square Error (RMSE), r², and p-value, are shown in FIG. 17C. These results support that Equation 4 predicts the actual (measured) FcγRIIIa binding with statistical significance. Higher levels of unpaired afucosylated glycans and unpaired high mannose glycans result in higher FcγRIIIa binding activity.

These results suggest that target FcγRIIIa binding levels of an antibody may be predominately reflected by the relative abundance of unpaired afucosylated glycans and/or the relative abundance of unpaired high mannose glycans of an antibody composition, and that target FcγRIIIa binding levels of an antibody may be modified by altering conditions to modify the relative abundance of unpaired afucosylated glycans and/or the relative abundance of unpaired high mannose glycans.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of producing an antibody composition, said method comprising:
i. determining the relative unpaired afucosylated glycan content and/or the relative unpaired high mannose glycan content of a sample of the antibody composition; and
ii. selecting the antibody composition for downstream processing based on the relative unpaired afucosylated glycan content and/or relative unpaired high mannose glycan content determined in (i).

2. The method of claim 1, wherein the sample is taken from a cell culture comprising cells expressing an antibody of the antibody composition.

3. The method of claim 2, further comprising modifying one or more conditions of the cell culture to modify the relative unpaired afucosylated glycan content and/or the relative unpaired high mannose glycan content of the antibody composition and determining the relative unpaired afucosylated glycan content and/or the relative unpaired high mannose glycan content of a sample of the modified cell culture.

4. The method of claim 3, comprising repeating the modifying until the relative unpaired afucosylated glycan content and/or relative unpaired high mannose glycan content is within a target range.

5. The method of any one of the preceding claims, wherein the relative unpaired afucosylated glycan content and/or the relative unpaired high mannose glycan content is/are determined in real time with respect to production of the antibody composition.

6. The method of claim of any one of the preceding claims, comprising selecting the antibody composition for downstream processing when the relative unpaired afucosylated glycan content and/or relative unpaired high mannose glycan content is/are in a target range.

7. The method of any one of the preceding claims, wherein the relative unpaired afucosylated glycan content and/or relative unpaired high mannose glycan content correlate with the ADCC activity level of the antibody composition.

8. The method of claim 7, further comprising determining the ADCC activity level of the antibody composition based on the relative unpaired afucosylated glycan content and/or relative unpaired high mannose glycan content determined in (i).

9. The method of claim 8, comprising selecting the antibody composition for downstream processing when the ADCC activity level is in a target range.

10. A method of producing an antibody composition, said method comprising:
i. determining the relative unpaired afucosylated glycan content of an antibody composition and/or the relative unpaired high mannose glycan content of an antibody composition;
ii. determining the ADCC level of the antibody composition based on the relative unpaired afucosylated glycan content and/or the relative unpaired high mannose glycan content determined in (i);
iii. selecting the antibody composition for downstream processing when the ADCC level of the antibody composition determined in (ii) is within a target ADCC range.

11. A method of producing an antibody composition, said method comprising:
i. determining the relative unpaired afucosylated glycan content and/or the relative unpaired high mannose glycan content of a sample of the antibody composition taken from a cell culture comprising glycosylation-competent cells expressing an antibody of the antibody composition;
ii. optionally,
modifying the cell culture to modulate the relative unpaired afucosylated glycan content and/or the relative unpaired high mannose glycan content, and
determining the relative unpaired afucosylated glycan content and/or the relative unpaired high mannose glycan content of a sample of the antibody composition taken from the modified cell culture;
iii. selecting the antibody composition for downstream processing based on the relative unpaired afucosylated glycan content and/or relative unpaired high mannose glycan content.

12. The method of any one of the preceding claims, further comprising maintaining a cell culture comprising glycosylation-competent cells expressing an antibody of the antibody composition.

13. The method of any one of the preceding claims, wherein the downstream processing comprises at least one of dilution, concentration, filling, filtration, formulation, chromatography, viral filtration, and/or viral inactivation.

14. The method of any one of the preceding claims, wherein the downstream processing comprises chromatography such as capture chromatography, intermediate chromatography, and/or polish chromatography.

15. The method of claim 14, wherein the chromatography comprises one or more of affinity chromatography, ion exchange chromatography, or hydrophobic interaction chromatography.

16. The method of any one of the preceding claims, wherein the determining of (i) comprises treating the antibody composition with an enzyme which cleaves an antibody heavy chain at a site N-terminal to the hinge region disulfide linkages to form antibody fragments, (ii) separating the antibody fragments by a chromatography, and (iii) quantifying each antibody fragment.

17. The method of claim 16, wherein the site is between Thr and His or between Lys and Thr of the sequence KTHTCPP (SEQ ID NO: 1) of an IgG1 antibody heavy chain.

18. The method of claim 16 or 17, wherein the antibody fragments comprises Fab fragments and glycosylated Fc fragments.

19. The method of any one of claims 16 to 18, wherein the separating of (ii) comprises hydrophilic interaction liquid chromatography (HILIC).

20. The method of any one of the preceding claims, wherein the quantifying of (iii) comprises mass spectrometry.

21. The method of any one of claims 18 to 20, wherein the glycosylated Fc fragments comprise Fc fragments attached to one of a variety of glycan moieties, and the method comprises separating and quantifying glycosylated Fc fragments according to the attached glycan moiety.

22. The method of any one of the preceding claims, wherein selecting the antibody composition for downstream processing comprises selecting a clone that produces the antibody composition having a selected relative unpaired afucosylated glycan content and/or relative unpaired high mannose glycan content.

23. A method of determining the relative unpaired glycan content of an antibody composition, comprising (i) treating the antibody composition with an enzyme which cleaves an antibody heavy chain at a site N-terminal to the hinge region disulfide linkages to form antibody fragments, (ii) separating the antibody fragments by a chromatography, and (iii) quantifying each antibody fragment.

24. The method of claim 23, wherein the site is between Thr and His or between Lys and Thr of the sequence KTHTCPP (SEQ ID NO: 1) of an IgG1 antibody heavy chain.

25. The method of claim 23 or 24, wherein the antibody fragments comprises Fab fragments and glycosylated Fc fragments.

26. The method of any one of claims 23 to 25, wherein the separating of (ii) comprises hydrophilic interaction liquid chromatography (HILIC).

27. The method of any one of claims 23 to 26, wherein the quantifying of (iii) comprises mass spectrometry.

28. The method of any one of claims 25 to 27, wherein the glycosylated Fc fragments comprise Fc fragments attached to one of a variety of glycan moieties, and the method comprises separating and quantifying glycosylated Fc fragments according to the attached glycan moiety.

29. The method of any one of claims 23 to 28, wherein relative unpaired afucosylated glycans and/or relative unpaired high mannose glycans of the antibody composition is/are quantified.

30. A method of modifying the ADCC level of an antibody composition, comprising modifying the relative unpaired afucosylated glycan content of an antibody composition and/or the relative unpaired high mannose glycan content of an antibody composition.

31. The method of claim 30, comprising increasing the relative unpaired afucosylated glycan content to increase the level of ADCC activity.

32. The method of claim 30 or 31, comprising increasing the relative unpaired high mannose glycan content to increase the level of ADCC activity.

33. The method of claim 30, comprising decreasing the relative unpaired afucosylated glycan content to decrease the level of ADCC activity.

34. The method of claim 30 or 33, comprising decreasing the relative unpaired high mannose glycan content to decrease the level of ADCC activity.

35. The method of claim 30-34, wherein the level of ADCC activity is modified by about 11% to about 14%, when the relative unpaired afucosylated glycan content and/or the relative unpaired high mannose glycan content is modified by about 1% and the antibody of the antibody composition is an anti-TNF antibody, optionally, infliximab.

36. The method of claim 30-35, wherein the level of ADCC activity is modified by about 11% to about 14%, when each of the relative unpaired afucosylated glycan content and the relative unpaired high mannose glycan content is modified by about 1% and the antibody of the antibody composition is an anti-TNF antibody, optionally, infliximab.

37. The method of any one of claims 30-36, wherein the level of ADCC activity is modified by about 13% to about 15%, when the relative unpaired afucosylated glycan content target range is modified by about 1% and the antibody is an anti-HER2 antibody, optionally, trastuzumab.

38. The method of any one of claims 1 to 29, comprising modifying the ADCC level of an antibody composition according to the method of any one of claims 30 to 37.
